(19) United States
(12) Patent Application Publication
(10) Pub. No.: US 2011/0014218 A1
(43) Pub. Date: Jan. 20, 2011

(54) IXODES SCAPULARIS SALIVARY PROTEINS AND METHODS OF USE FOR MODULATION OF THE ALTERNATIVE COMPLEMENT PATHWAY

(75) Inventors: Aravinda De Silva, Chapel Hill, NC (US); Katharine Tyson, Hillsborough, NC (US); Erol Fikrig, Guilford, CT (US)

Correspondence Address: JENKINS, WILSON, TAYLOR & HUNT, P. A., 3100 Tower Blvd., Suite 1200, DURHAM, NC, 27707, US

(73) Assignee: University of North Carolina at Chapel Hill

(21) Appl. No.: 12/811,309

(22) PCT Filed: Jan. 26, 2009
(86) PCT No.: PCT/US09/32004

§ 371 Date: Oct. 1, 2010

Related U.S. Application Data
(60) Provisional application No. 61/062,303, filed on Jan. 25, 2008.

(51) Int. Cl. A61K 39/00; C07K 14/00; A61K 38/16; C07H 21/04; C12N 15/63; C12N 5/10; G01N 33/53; A61P 37/02

(52) U.S. Cl. 424/185.1; 530/350; 514/21.2; 536/23.5; 435/320.1; 435/325; 436/501

(57) ABSTRACT

Ixodes Scapularis salivary proteins, including Ixodes Scapularis anti-complement protein (Isac) and Isac-like protein family (ILP family) proteins, biologically functional equivalents and fragments thereof, and nucleic acid molecules encoding the same are disclosed. ILP family proteins, gene products and polypeptide fragments bind to proteins with thrombospondin repeats. Thus, therapeutic methods involving modulating proteins with thrombospondin repeats using ILP family proteins and biologically active polypeptide fragments thereof are also disclosed. ILP family proteins, gene products and polypeptide fragments have biological activity in modulating the complement pathway through specific binding to properdin. Thus, therapeutic methods involving modulating the complement pathway using ILP family proteins and biologically active polypeptide fragments thereof are also disclosed. The specific binding of ILP family proteins to properdin also provides for methods of treating conditions associated with inappropriate complement pathway activation. Screening methods for selecting substances having an ability to bind to proteins with thrombospondin repeats, including properdin, are also disclosed. Screening methods for selecting substances having an ability to modulate complement pathway activity are also disclosed.

### RELATED APPLICATIONS

The presently disclosed subject matter claims the benefit of U.S. Provisional Patent Application Ser. No. 61/062,303, filed Jan. 25, 2008; the disclosure of which is incorporated herein by reference in its entirety.

### GOVERNMENT INTEREST

This presently disclosed subject matter was made with U.S. Government support under Grant No. 1U01AI058263 awarded by National Institutes of Health. Thus, the U.S. Government has certain rights in the presently disclosed subject matter.

### TECHNICAL FIELD

The presently disclosed subject matter relates generally to isolated and purified polypeptides and nucleic acids and methods of using same. More particularly, the presently disclosed subject matter relates to isolated and purified members of the Ixodes Scapularis anti-complement protein (Isac) and Isac-like protein family (ILP family), and purified nucleic acid molecules encoding same. The presently disclosed subject matter further relates to methods of using the polypeptides to modulate the alternative complement pathway, including therapeutic methods for treating disorders related to inappropriate complement pathway activation. The presently disclosed subject matter further relates to methods of modulating the activity of proteins with thrombospondin repeats. The presently disclosed subject matter further relates to screening methods for selecting compositions that can modulate the alternative complement pathway, properdin binding and/or the activity of proteins with thrombospondin repeats.

### BACKGROUND

The immune system is highly complex and tightly regulated, with many alternative pathways capable of compensating for deficiencies in other parts of the system. The complement pathway of the immune system is integral to effective immune function. When functioning correctly, the complement pathway works in concert with antibodies and the rest of the immune system to maximize immunological protection. However, when improperly activated, the immune response can become a cause of disease or other undesirable conditions. In particular, the complement pathway of the immune system, including the alternative complement pathway, can create health risks, particularly those associated with inflammation, when improperly activated. As such, there exists a need for the ability to modulate or suppress the complement pathway.

Ixodes scapularis ticks produce salivary proteins with the ability to modulate the host immune response. In particular, it is important for the parasite I. scapularis to prevent host inflammation and immune recognition at the feeding site. Thus, it appears that the salivary proteins produced by I. scapularis ticks display unique characteristics that inhibit the alternative complement pathway. Therefore, identifying novel I. scapularis salivary proteins and further defining their functional characteristics can provide the ability to modulate the complement pathway.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In one embodiment, the presently disclosed subject matter provides an isolated and purified ILP family polypeptide, comprising (a) a polypeptide encoded by a nucleic acid sequence of any of odd numbered SEQ ID NOs: 1-30, (b) polypeptide encoded by a nucleic acid having at least about 90% or greater sequence identity to a DNA sequence of any of odd numbered SEQ ID NOs: 1-30, (c) a polypeptide having an amino acid sequence of any of even numbered SEQ ID NOs: 1-30, or (d) a polypeptide having an amino acid sequence having at least about 90% or greater sequence identity to an amino acid sequence of any of even numbered SEQ ID NOs: 1-30. In some embodiments, the polypeptide is modified to be in detectably labeled form. In some embodiments, a composition comprising the polypeptide and a carrier is provided. In some embodiments, the carrier is a pharmaceutically acceptable carrier.

In some embodiments of the presently disclosed subject matter, an isolated nucleic acid molecule is provided, comprising (a) a nucleic acid molecule encoding a polypeptide of any of even numbered SEQ ID NOs: 1-30, (b) a nucleic acid molecule encoding a polypeptide having at least about 90% or greater sequence identity to a polypeptide of any of even numbered SEQ ID NOs: 1-30, (c) a nucleic acid molecule having at least about 90% or greater sequence identity to a nucleic acid sequence of any of odd numbered SEQ ID NOs: 1-30, or (d) a nucleic acid molecule having a nucleic acid sequence of any of odd numbered SEQ ID NOs: 1-30. In some embodiments, a recombinant vector comprising the isolated nucleic acid molecule operatively linked to a promoter is provided, and in some embodiments a recombinant host cell comprising the nucleic acid molecule is further provided.

In some embodiments the presently disclosed subject matter provides a method of modulating the activity of a protein having thrombospondin repeats, comprising contacting the protein having thrombospondin repeats with an ILP family protein comprising (a) a polypeptide encoded by a nucleic acid sequence of any of odd numbered SEQ ID NOs: 1-36, (b) a polypeptide encoded by a nucleic acid having at least about 90% or greater sequence identity to a DNA sequence of any of odd numbered SEQ ID NOs: 1-36, (c) a polypeptide having an amino acid sequence of any of even numbered SEQ ID NOs: 1-36, or (d) a polypeptide having an amino acid sequence having at least about 90% or greater sequence identity to an amino acid sequence of any of even numbered SEQ ID NOs: 1-36, wherein activity of the protein having thrombospondin repeats is modulated. In some embodiments, the protein having thrombospondin repeats is properdin. In some embodiments, the thrombospondin repeats are type 1 thrombospondin repeats. In some embodiments, the protein having thrombospondin repeats is selected from a protein involved in cancer, homeostasis and pathogenesis. In some embodiments, the protein having thrombospondin repeats is within a subject and the ILP family protein is administered to the subject. In some embodiments, the ILP family protein is administered by systemic administration, parenteral administration, intravascular administration, intramuscular administration, intraarterial administration, oral delivery, buccal delivery, subcutaneous administration, inhalation, intratracheal installation, surgical implantation, transdermal delivery, local injection, hyper-velocity injection/bombardment, or combinations thereof.

In some embodiments, the presently disclosed subject matter provides a method of modulating the alternative complement pathway in a subject, comprising administering to the subject an effective amount of an ILP family protein comprising (a) a polypeptide encoded by a nucleic acid sequence of any of odd numbered SEQ ID NOs: 1-36, (b) a polypeptide encoded by a nucleic acid having at least about 90% or greater sequence identity to a DNA sequence of any of odd numbered SEQ ID NOs: 1-36, (c) a polypeptide having an amino acid sequence of any of even numbered SEQ ID NOs: 1-36, or (d) a polypeptide having an amino acid sequence having at least about 90% or greater sequence identity to an amino acid sequence of any of even numbered SEQ ID NOs: 1-36, wherein the alternative complement pathway is modulated. In some embodiments, the method comprises reducing the activity of the alternative complement pathway. In some embodiments, reducing the activity of the alternative complement pathway comprises the binding of the ILP family protein to properdin thereby accelerating the decay of the C3 convertase and reducing the activity of the alternative complement pathway. In some embodiments, ILP family protein binds to properdin by binding to the thrombospondin repeats on properdin. In some embodiments, the ILP family protein is administered by systemic administration, parenteral administration, intravascular administration, intramuscular administration, intraarterial administration, oral delivery, buccal delivery, subcutaneous administration, inhalation, intratracheal installation, surgical implantation, transdermal delivery, local injection, hyper-velocity injection/bombardment, or combinations thereof. In some embodiments, the subject is suffering from a condition associated with inappropriate alternative complement pathway activation. In some embodiments, the condition associated with inappropriate complement pathway activation is selected from inflammatory diseases, arthritis, asthma, acute injuries, burns, heart disease, autoimmune diseases and SARS.

In some embodiments, the presently disclosed subject matter provides a method of treating a complication associated with inappropriate alternative complement pathway activation in a subject, comprising administering to the subject an effective amount of an ILP family protein comprising (a) a polypeptide encoded by a nucleic acid sequence of any of odd numbered SEQ ID NOs: 1-36, (b) a polypeptide encoded by a nucleic acid having at least about 90% or greater sequence identity to a DNA sequence of any of odd numbered SEQ ID NOs: 1-36, (c) a polypeptide having an amino acid sequence of any of even numbered SEQ ID NOs: 1-36, or (d) a polypeptide having an amino acid sequence having at least about 90% or greater sequence identity to an amino acid sequence of any of even numbered SEQ ID NOs: 1-36, wherein the complication is treated. In some embodiments, the method comprises reducing the activity of the alternative complement pathway. In some embodiments, the method comprises binding of the ILP family protein to properdin thereby accelerating the decay of the C3 convertase and reducing the activity of the alternative complement pathway. In some embodiments, the ILP family protein binds to properdin by binding to the thrombospondin repeats on properdin. In some embodiments, the ILP family protein is administered by systemic administration, parenteral administration, intravascular administration, intramuscular administration, intraarterial administration, oral delivery, buccal delivery, subcutaneous administration, inhalation, intratracheal installation, surgical implantation, transdermal delivery, local injection, hyper-velocity injection/bombardment, or combinations thereof. In some embodiments, the complication associated with inappropriate alternative complement pathway activation is selected from inflammatory diseases, arthritis, asthma, acute injuries, burns, heart disease, autoimmune diseases and SARS.

In some embodiments, the presently disclosed subject matter provides a method of screening a candidate substance for an ability to bind properdin, the method comprising (a) establishing a test sample comprising properdin, (b) administering a candidate substance to the test sample, and (c) determining the ability of the candidate substance to bind to properdin. In some embodiments, the method further comprises administering an ILP family protein to the test sample in step (b) and determining the ability of the candidate substance to bind to properdin based upon the competition between the candidate substance and the ILP family protein in step (c).

In some embodiments, the presently disclosed subject matter provides a method of screening for substances capable of modulating the activity of the alternative complement pathway, comprising (a) establishing a test sample comprising a protein having thrombospondin repeats, (b) administering a candidate substance to the test sample, (c) determining the ability of the candidate substance to bind to the protein having thrombospondin repeats, and (d) identifying a candidate substance as capable of inhibiting the alternative complement pathway where the candidate substance is capable of binding to the protein having thrombospondin repeats. In some embodiments, the protein having thrombospondin repeats is properdin. In some embodiments, the method further comprises administering an ILP family protein to the test sample in step (b) and determining the ability of the candidate substance to bind to the protein having thrombospondin repeats based upon the competition between the candidate substance and the ILP family protein in step (c)

Therefore, it is an object of the presently disclosed subject matter to provide compositions comprising ILP family polypeptides, and related methods of making and using the same.

An object of the presently disclosed subject matter having been stated hereinabove, and which is addressed in whole or in part by the presently disclosed subject matter, other objects will become evident as the description proceeds when taken in connection with the accompanying drawings and examples as best described hereinbelow.

### BRIEF DESCRIPTION OF THE DRAWINGS

FIG. 1 is an amino acid alignment of ILP family proteins, including Salp20, Isac, and novel cDNA clones.

FIG. 2 is a line graph showing that the ILP family protein Salp20, also referred to as S20NS, inhibits the alternative complement pathway by dissociating the C3 convertase.

FIGS. 3A-3C show that S20NS does not dissociate the C3 convertase by a mechanism similar to fH or fI.

FIG. 3A is a bar graph showing the dissociation of Bb from C3 convertase by S20NS.

FIG. 3B is an autoradiograph showing the inability of S20NS to mediate fI degradation of C3b.

FIG. 3C is an autoradiograph showing the degradation of C3b where S20NS or fI were incubated with C3b in the presence of fH.

FIG. 4 is a bar graph showing that S20NS dissociates the C3 convertase only in the presence of properdin.

FIGS. 5A-5C show that S20NS dissociates properdin from the C3 convertase.

FIG. 5A is a bar graph showing properdin displacement from C3 convertase by S20NS.

FIG. 5B is a bar graph showing S20NS mediated properdin displacement from C3 convertase formed from NHS.

FIG. 5C is a bar graph showing S20NS mediated properdin displacement from 03 convertase containing only C3bP.

FIGS. 6A-6C show that S20NS binds properdin.

FIG. 6A is an autoradiograph of a Western blot showing direct binding of properdin by S20NS.

FIG. 6B is a bar graph showing specific binding of properdin by S20NS.

FIG. 6C is a line graph showing the relative affinity of properdin for S20NS or C3b.

### BRIEF DESCRIPTION OF THE SEQUENCE LISTING

SEQ ID NO: 1 is a polynucleotide sequence encoding an Ixodes scapularis anti-complement protein (Isac)-like protein (ILP) polypeptide isolated from Ixodes scapularis.

SEQ ID NO: 2 is an ILP polypeptide sequence encoded by SEQ ID NO: 1, also referred to herein as S20Lclone 1 or Salp20-like protein 1.

SEQ ID NO: 3 is a polynucleotide sequence encoding an ILP polypeptide isolated from Ixodes scapularis.

SEQ ID NO: 4 is an ILP polypeptide sequence encoded by SEQ ID NO: 3, also referred to herein as S20Lclone 2 or Salp20-like protein 2.

SEQ ID NO: 5 is a polynucleotide sequence encoding an ILP polypeptide isolated from Ixodes scapularis.

SEQ ID NO: 6 is an ILP polypeptide sequence encoded by SEQ ID NO: 5, also referred to herein as S20Lclone 3 or Salp20-like protein 3.

SEQ ID NO: 7 is a polynucleotide sequence encoding an ILP polypeptide isolated from Ixodes scapularis.

SEQ ID NO: 8 is an ILP polypeptide sequence encoded by SEQ ID NO: 7, also referred to herein as S20Lclone 4 or Salp20-like protein 4.

SEQ ID NO: 9 is a polynucleotide sequence encoding an ILP polypeptide isolated from Ixodes scapularis.

SEQ ID NO: 10 is an ILP polypeptide sequence encoded by SEQ ID NO: 9, also referred to herein as S20Lclone 5 or Salp20-like protein 5.

SEQ ID NO: 11 is a polynucleotide sequence encoding an ILP polypeptide isolated from Ixodes scapularis.

SEQ ID NO: 12 is an ILP polypeptide sequence encoded by SEQ ID NO: 11, also referred to herein as S20Lclone 6 or Salp20-like protein 6.

SEQ ID NO: 13 is a polynucleotide sequence encoding an ILP polypeptide isolated from Ixodes scapularis.

SEQ ID NO: 14 is an ILP polypeptide sequence encoded by SEQ ID NO: 13, also referred to herein as S20Lclone 7 or Salp20-like protein 7.

SEQ ID NO: 15 is a polynucleotide sequence encoding an ILP polypeptide isolated from Ixodes scapularis.

SEQ ID NO: 16 is an ILP polypeptide sequence encoded by SEQ ID NO: 15, also referred to herein as S20Lclone 8 or Salp20-like protein 8.

SEQ ID NO: 17 is a polynucleotide sequence encoding an ILP polypeptide isolated from Ixodes scapularis.

SEQ ID NO: 18 is an ILP polypeptide sequence encoded by SEQ ID NO: 17, also referred to herein as S20Lclone 9 or Salp20-like protein 9.

SEQ ID NO: 19 is a polynucleotide sequence encoding an ILP polypeptide isolated from Ixodes scapularis.

SEQ ID NO: 20 is an ILP polypeptide sequence encoded by SEQ ID NO: 19, also referred to herein as S20Lclone 10 or Salp20-like protein 10.

SEQ ID NO: 21 is a polynucleotide sequence encoding an ILP polypeptide isolated from Ixodes scapularis.

SEQ ID NO: 22 is an ILP polypeptide sequence encoded by SEQ ID NO: 21, also referred to herein as S20Lclone 11 or Salp20-like protein 11.

SEQ ID NO: 23 is a polynucleotide sequence encoding an ILP polypeptide isolated from Ixodes scapularis.

SEQ ID NO: 24 is an ILP polypeptide sequence encoded by SEQ ID NO: 23, also referred to herein as S20Lclone 12 or Salp20-like protein 12.

SEQ ID NO: 25 is a polynucleotide sequence encoding an ILP polypeptide isolated from Ixodes scapularis.

SEQ ID NO: 26 is an ILP polypeptide sequence encoded by SEQ ID NO: 25, also referred to herein as S20Lclone 13 or Salp20-like protein 13.

SEQ ID NO: 27 is a polynucleotide sequence encoding an ILP polypeptide isolated from Ixodes scapularis.

SEQ ID NO: 28 is an ILP polypeptide sequence encoded by SEQ ID NO: 27, also referred to herein as S20Lclone 14 or Salp20-like protein 14.

SEQ ID NO: 29 is a polynucleotide sequence encoding an ILP polypeptide isolated from Ixodes scapularis.

SEQ ID NO: 30 is an ILP polypeptide sequence encoded by SEQ ID NO: 29, also referred to herein as S20Lclone 15 or Salp20-like protein 15.

SEQ ID NO: 31 is a polynucleotide sequence encoding Ixodes scapularis anti-complement protein (Isac) isolated from Ixodes scapularis.

SEQ ID NO: 32 is the Isac polypeptide sequence encoded by SEQ ID NO: 31.

SEQ ID NO: 33 is a polynucleotide sequence encoding an ILP polypeptide isolated from Ixodes scapularis.

SEQ ID NO: 34 is an ILP polypeptide sequence encoded by SEQ ID NO: 33, also referred to as Salp9.

SEQ ID NO: 35 is a polynucleotide sequence encoding an ILP polypeptide isolated from Ixodes scapularis.

SEQ ID NO: 36 is an ILP polypeptide sequence encoded by SEQ ID NO: 35, also referred to as Salp20.

SEQ ID NO: 37 is an oligonucleotide primer used to amplify Salp20 by PCR, also referred to as KS20F.

SEQ ID NO: 38 is an oligonucleotide primer used to amplify Salp20 by PCR, also referred to as S20R.

SEQ ID NO: 39 is an oligonucleotide primer used to amplify Isac by PCR, also referred to as Isac F.

SEQ ID NO: 40 is an oligonucleotide primer used to amplify Isac by PCR, also referred to as Isac R.

### DETAILED DESCRIPTION

The presently disclosed subject matter provides compositions capable of specifically binding to proteins having thrombospondin repeats. In some embodiments, the presently disclosed subject matter provides Ixodes Scapularis anti-complement protein (Isac) and Isac-like protein family (ILP family) proteins capable of binding properdin and of modulating the alternative complement pathway. Compositions containing ILP family proteins can be used to treat conditions associated with inappropriate complement pathway activation. In some embodiments, the compositions disclosed herein are further useful in methods of screening for additional substances having similar properties as the presently disclosed compositions.

In some embodiments, the presently disclosed compositions, which are capable of specifically binding to and modulating the ability of properdin to act as a positive regulator of the complement pathway, comprise ILP family proteins, or biologically active fragments thereof. Representative ILP family proteins set forth in SEQ ID NOs: 1-30, disclosed herein for the first time, inhibit the activation and/or activity of the alternative complement pathway. It has also been discovered, and disclosed herein for the first time, that the inhibition of the alternative complement pathway by ILP family proteins can be exerted through a direct and specific association between the ILP family protein and the thrombospondin repeats on properdin. The direct binding of an ILP family protein to properdin displaces the properdin from the C3 convertase, thereby causing the accelerated decay of the C3 convertase and subsequent decreased activity of the alternative complement pathway. Thus, the presently disclosed subject matter provides novel ILP family proteins that have an immunosuppressive effect on the complement pathway by way of a novel mechanism, as disclosed in detail herein.

### I. General Considerations

The complement system is made up of a series of about 25 proteins that work to “complement” the activity of antibodies in destroying bacteria, either by facilitating phagocytosis or by puncturing the bacterial cell membrane. Complement also helps to rid the body of antigen-antibody complexes. In carrying out these tasks, it induces an inflammatory response. Complement proteins circulate in the blood in an inactive form. When the first of the complement substances is triggered, usually by an antibody interlocked with an antigen, it initiates a cascade of downstream reactions involving multiple components of the complement system. As each component is activated in turn, it acts upon the next in a precise sequence of carefully regulated steps known as the “complement cascade”.

Complement activation occurs by two different sequences, the classic and alternative pathways. The components within each complement cascade vary between the classical and alternative pathways. In general, the classic pathway is activated by the binding of the C1 component to classic pathway activators, primarily antigen-antibody complexes containing IgM, IgG1, and IgG3, while the alternative pathway can be activated by IgA immune complexes and also by nonimmunologic materials including bacterial endotoxins, microbial polysaccharides and cell walls.

Both pathways end in creation of a unit known as the membrane attack complex. Inserted in the wall of the target cell, the membrane attack complex constitutes a channel which disrupts the integrity of the cell membrane and causes the target cell to rapidly swell and burst.

By way of elaboration, the alternative pathway of complement is activated when C3b binds covalently through its reactive thioester to activating surfaces (Walport, 2001). Surface bound C3b binds factor B, which is then cleaved by factor D, producing the cleavage products Bb and Ba. Bb remains bound to C3b, while Ba is released. The surface bound C3bBb complex, or C3 convertase, cleaves additional C3 components producing more C3b that either binds to activating surfaces or to the C3 convertase, forming the C5 convertase. The C5 convertase then initializes the formation of the membrane attack complex. The alternative complement pathway can be initiated by metastable C3(H₂O), a naturally occurring hydrolysed C3 molecule. C3(H₂O) resembles C3b and binds fB in solution, allowing fB to then be cleaved by fD. The resulting fluidphase convertase, C3(H₂O)Bb, then cleaves C3, releasing C3b that deposits onto surfaces activating the complement cascade (Pangburn et al., 1981).

Properdin is a protein of the alternative complement pathway. In particular, properdin is a positive regulator of complement activation that binds and stabilizes the C3bBb complex, or C3 convertase (Hourcade, 2006). Properdin comprises six type-1 thrombospondin like repeats, with each repeat carrying a separate ligand-binding site (Hourcade, 2006; Tyson et al., 2007). Previous reports suggest that properdin function can depend on multiple interactions between its subunits with its ligands (Hourcade, 2006).

Even though properdin is not an active component of the C3 convertase, it is essential for the stabilization and full activity of the convertase (Fearon et al., 1975; Gupta-Bansal et al., 2000). Gupta-Bansal et al. and Perdikoulis et al. have demonstrated that antibodies directed against properdin are capable of inhibiting the alternative pathway (Gupta-Bansal et al., 2000; Perdikoulis et al., 2001). Recent studies have also shown that properdin is capable of binding to cell surfaces and initiating the alternative pathway by providing a platform for the assembly of the C3 convertase (Spitzer et al., 2007). Since properdin plays a role in effective complement activation, it is an attractive target for inactivation by pathogens or blood feeding organisms. One example of a virulence factor that targets properdin is streptococcal pyrogenic exotoxin B, which acts to degrade properdin, allowing the pathogenic group A streptococci to resist opsonophagocytosis mediated by complement (Spitzer et al., 2007).

In addition to mediating lysis and opsonization of invading pathogens, the alternative complement cascade also leads to the production of anaphylatoxins, which are proinflammatory mediators that recruit neutrophils and monocytes to the site of complement activation (Walport, 2001). As such, activation of the complement pathway, including the alternative complement pathway, causes inflammation. Thus, when the alternative complement pathway is inappropriately activated it can cause tissue damage due to its proinflammatory effect. Therefore, there exists a need for methods to suppress the alternative complement pathway in the event of inappropriate activation.

In order to thrive in nature Ixodes scapularis ticks are able to modulate the host immune response. Inhibition of the alternative complement pathway by I. scapularis is important for preventing host inflammation and immune recognition at the feeding site, thereby allowing the tick to feed successfully to repletion. In addition, inhibition of the alternative complement pathway by tick saliva during feeding potentially allows the successful transmission of pathogens throughout the feeding period of 5 days. I. scapularis ticks act as the vector for several pathogens including the causative agents of Lyme disease and human granulocytic ehrlichiosis (Burgdorfer et al., 1982; Chen et al., 1994). Tick salivary proteins enter the host during feeding and exert pleiotropic immunosuppressive effects (Anguita et al., 2002; Ferreira and Silva, 1998; Kopecky and Kuthejlova, 1998; Ribeiro et al., 1995; Schoeler et al., 1999; Urioste et al., 1994; Wikel and Bergman, 1997).

Ixodes scapularis salivary protein, also referred to as I. scapularis anticomplement protein (Isac), with a predicted mass of 18 kDa, inhibits the alternative pathway of complement. At least one mechanism by which Isac is believed to exert its inhibitory effect on Isac is by dissociating the components of the C3 convertase and preventing the deposition of C3b onto surfaces, similar to factor H and factor H-like protein 1 (Valenzuela et al., 2000). Two other I. scapularis salivary proteins, I. scapularis salivary protein 9 (Salp9) and I. scapularis salivary protein 20 (Salp20), which share homology with Isac, have been identified. These three salivary proteins, Salp9, Salp20, and Isac, identified by Soares at al. and Ribeiro et al., are included in a large family of related I. scapularis salivary anticomplement proteins, or Isac-like protein family (ILP family). Disclosed herein are fifteen (15) novel ILP family proteins.

The mechanism(s) by which each of the previously identified ILP family proteins inhibits the alternative pathway has yet to be completely elucidated. In particular, as described herein, ILP family proteins, including those disclosed herein for the first time, are candidates for use in immunosuppressive therapies. A clear understanding of the mechanism by which ILP family proteins cause immunosuppression, however, is needed for continuing further studies regarding its potential use. Prior to the discovery of the subject matter disclosed herein, a full understanding of ILP family protein mechanisms of immunosuppression was unknown.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage can encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The term “antibody” or “antibody molecule” refers collectively to a population of immunoglobulin molecules and/or immunologically active portions of immunoglobulin molecules, i.e., molecules that contain a paratope. A paratope is the portion or portions of antibodies that is or are responsible for that antibody binding to an antigenic determinant, or epitope.

Representative antibodies for use in the present subject matter are intact immunoglobulin molecules, substantially intact immunoglobulin molecules, single chain immunoglobulins or antibodies, those portions of an immunoglobulin molecule that contain the paratope, including antibody fragments. A monovalent antibody can optionally be used.

The terms “associated with”, “operably linked”, and “operatively linked” refer to two nucleic acid sequences that are related physically or functionally. For example, a promoter or regulatory DNA sequence is said to be “associated with” a DNA sequence that encodes an RNA or a polypeptide if the two sequences are operatively linked, or situated such that the regulator DNA sequence will affect the expression level of the coding or structural DNA sequence.

The terms “C3 convertase”, “C3”, “C3bBb complex” and “C3bP complex” are used interchangeably herein and refer to an intermediary complex in the alternative complement pathway to which properdin binds and acts as a positive regulator of the complement pathway. The C3 convertase can comprise the components C3b and Bb. By way of elaboration, the alternative pathway of complement is activated when C3b binds covalently through its reactive thioester to activating surfaces (Walport, 2001). Surface bound C3b binds factor B, which is then cleaved by factor D, producing the cleavage products Bb and Ba. Bb remains bound to C3b, while Ba is released. The surface bound C3bBb complex, or C3 convertase, cleaves additional C3 components producing more C3b that either binds to activating surfaces or to the C3 convertase, forming the C5 convertase. The C5 convertase then initializes the formation of the membrane attack complex. Properdin can bind directly to the C3 convertase and provide stability, thereby increasing the half-life of C3 convertase. Displacement of properdin from C3 convertase by an ILP family protein binding to the properdin can accelerate the decay of the C3 convertase, thereby inhibiting activation of and/or activity of the complement pathway.

The terms “coding sequence” and “open reading frame” (ORF) are used interchangeably and refer to a nucleic acid sequence that is transcribed into RNA such as mRNA, rRNA, tRNA, snRNA, shRNA, siRNA, sense RNA, or antisense RNA. In some embodiments, the RNA is then translated in vivo or in vitro to produce a polypeptide.

The term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction.

The terms “complement”, “complement pathway” and “complement system” are used interchangeably herein and refer to the complement system of a subject's immune system. The complement pathway is made up of a series of about 25 proteins that work to “complement” the activity of antibodies in destroying bacteria, either by facilitating phagocytosis or by puncturing the bacterial cell membrane. Complement also helps to rid the body of antigen-antibody complexes. In carrying out these tasks, it induces an inflammatory response. Complement proteins circulate in the blood in an inactive form. When the first of the complement substances is triggered, usually by an antibody interlocked with an antigen, it initiates a cascade of downstream reactions involving multiple components of the complement system. As each component is activated in turn, it acts upon the next in a precise sequence of carefully regulated steps known as the “complement cascade”.

Complement activation occurs by two different sequences, the classic and alternative pathways. The components within each complement cascade vary between the classical and alternative pathways. In general, the classic pathway is activated by the binding of the Cl component to classic pathway activators, primarily antigen-antibody complexes containing IgM, IgG1, and IgG3, while the alternative pathway can be activated by IgA immune complexes and also by nonimmunologic materials including bacterial endotoxins, microbial polysaccharides and cell walls.

Both pathways end in creation of a unit known as the membrane attack complex. Inserted in the wall of the target cell, the membrane attack complex constitutes a channel which disrupts the integrity of the cell membrane and causes the target cell to rapidly swell and burst.

The terms “alternative complement”, “alternative complement pathway”, “alternative pathway” and “alternative complement system” are used interchangeably herein to refer to the alternative complement pathway of the complement system.

The term “fragment” refers to a sequence that comprises a subset of another sequence. When used in the context of a nucleic acid or amino acid sequence, the terms “fragment” and “subsequence” are used interchangeably. A fragment of a nucleic acid sequence can be any number of nucleotides that is less than that found in another nucleic acid sequence, and thus includes, but is not limited to, the sequences of an exon or intron, a promoter, an enhancer, an origin of replication, a 5′ or 3′ untranslated region, a coding region, and a polypeptide binding domain. It is understood that a fragment or subsequence can also comprise less than the entirety of a nucleic acid sequence, for example, a portion of an exon or intron, promoter, enhancer, etc. Similarly, a fragment or subsequence of an amino acid sequence can be any number of residues that is less than that found in a naturally occurring polypeptide, and thus includes, but is not limited to, domains, features, repeats, etc. Also similarly, it is understood that a fragment or subsequence of an amino acid sequence need not comprise the entirety of the amino acid sequence of the domain, feature, repeat, etc.

A fragment can also be a “functional fragment”, in which the fragment retains a specific biological function of the nucleic acid sequence or amino acid sequence of interest. By way of example and not limitation, a functional fragment of an ILP family polypeptide can include a region having binding specificity for properdin and/or capable of modulating the alternative complement pathway.

The term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

The terms “heterologous”, “recombinant”, and “exogenous”, when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides.

A “homologous” nucleic acid (or amino acid) sequence is a nucleic acid (or amino acid) sequence naturally associated with a host cell into which it is introduced.

The term “inhibitor” refers to a chemical substance that inactivates or decreases the biological activity of a target entity such as a complement component. The term “isolated”, when used in the context of an isolated DNA molecule or an isolated polypeptide, is a DNA molecule or polypeptide that, by the hand of man, exists apart from its native environment and is therefore not a product of nature. An isolated DNA molecule or polypeptide can exist in a purified form or can exist in a non-native environment such as, for example, in a transgenic host cell.

As used herein, the term “modulate” means an increase, decrease, or other alteration of any, or all, chemical and biological activities or properties of a target entity, such as a wild-type or mutant polypeptide, including but not limited to a ILP family protein. The term “modulation” as used herein refers to both upregulation (i.e., activation or stimulation) and downregulation (i.e. inhibition or suppression) of a response, such as for example, modulation of the complement pathway, including modulation of activation of the complement pathway by binding or displacing properdin from the C3 complex.

As used herein, the term “mutation” carries its traditional connotation and means a change, inherited, naturally occurring or introduced, in a nucleic acid or polypeptide sequence, and is used in its sense as generally known to those of skill in the art.

The term “transformation” refers to a process for introducing heterologous DNA into a cell. Transformed cells are understood to encompass not only the end product of a transformation process, but also transgenic progeny thereof.

The terms “transformed”, “transgenic”, and “recombinant” refer to a cell of a host organism such as a mammal into which a heterologous nucleic acid molecule has been introduced. The nucleic acid molecule can be stably integrated into the genome of the cell or the nucleic acid molecule can also be present as an extrachromosomal molecule. Such an extrachromosomal molecule can be auto-replicating. Transformed cells, tissues, or subjects are understood to encompass not only the end product of a transformation process, but also transgenic progeny thereof. A “non-transformed,” “non-transgenic”, or “non-recombinant” host refers to a wild type organism, e.g., a mammal or a cell therefrom, which does not contain the heterologous nucleic acid molecule.

As used herein, the phrase “treating” refers to both intervention designed to ameliorate a condition in a subject (e.g., after initiation of a disease process or after an injury) as well as to interventions that are designed to prevent the condition from occurring in the subject. Stated another way, the terms “treating” and grammatical variants thereof are intended to be interpreted broadly to encompass meanings that refer to reducing the severity of and/or to curing a condition, as well as meanings that refer to prophylaxis. In this latter respect, “treating” can refer to “preventing” to any degree, or otherwise enhancing the ability of the subject to resist the process of the condition.

### III. Polypeptides and Nucleic Acids

The presently disclosed subject matter discloses isolated and purified biologically active ILP family polypeptides and nucleic acid molecules encoding same. As used in the following detailed description and in the claims, the term “ILP family peptide”, “ILP family protein”, “ILP family polypeptide” or “ILP family protein gene product” includes Ixodes scapularis anti-complement proteins (Isac) and Isac-like proteins (ILP), and biologically functional equivalents thereof and nucleic acids encoding same. The term “ILP family protein” includes homologs from non-tick species. Preferably, ILP family nucleic acids and polypeptides are isolated from eukaryotic sources.

The terms “ILP family protein gene product”, “ILP family protein”, “ILP family peptide”, “ILP family polypeptide”, “I. scapularis salivary protein”, and “tick salivary protein” refer to peptides having amino acid sequences which are substantially identical to native amino acid sequences from the organism of interest and which are biologically active in that they comprise all or a part of the amino acid sequence of an ILP family protein, or cross-react with antibodies raised against an ILP family protein, or retain all or some of the biological activity of the native amino acid sequence or protein. In some embodiments, an ILP family protein is a polypeptide isolated originally as a secreted salivary protein from Ixodes scapularis and set forth herein as any of the even numbered SEQ ID NOs: 2-36 and encoded by a polynucleotide as set forth herein as any of the odd numbered SEQ ID NOs:1-35. In some embodiments, an ILP family protein can be Isac. In some embodiments, an ILP family protein can be Salp20. In some embodiments, an ILP family protein can be Salp9. In some embodiments, an ILP family protein can be selected from the group including but not limited to Salp20-like protein 1 (SEQ ID NOs: 1, 2), Salp20-like protein 2 (SEQ ID NOs: 3, 4), Salp20-like protein 3 (SEQ ID NOs: 5, 6), Salp20-like protein 4 (SEQ ID NOs: 7, 8), Salp20-like protein 5 (SEQ ID NOs: 9, 10), Salp20-like protein 6 (SEQ ID NOs: 11, 12), Salp20-like protein 7 (SEQ ID NOs: 13, 14), Salp20-like protein 8 (SEQ ID NOs: 15, 16), Salp20-like protein 9 (SEQ ID NOs: 17, 18), Salp20-like protein 10 (SEQ ID NOs: 19, 20), Salp20-like protein 11 (SEQ ID NOs: 21, 22), Salp20-like protein 12 (SEQ ID NOs: 23, 24), Salp20-like protein 13 (SEQ ID NOs: 25, 26), Salp20-like protein 14 (SEQ ID NOs: 27, 28), and Salp20-like protein 15 (SEQ ID NOs: 29, 30), as set forth in Table 1.

In some embodiments, an ILP family polypeptide is modified to be in a detectably labeled form. A labeled form of an ILP family polypeptide has several utilities, as would be appreciated by one of skill in the art. For example, a labeled ILP family polypeptide could be used to identify the presence of a molecule to which an ILP family polypeptide binds with specificity in a sample, e.g., properdin. The molecule to which an ILP family polypeptide binds could be soluble or bound. For example, the molecule could be expressed by a cell, or certain types of cells, and a labeled ILP family polypeptide could be utilized to determine whether a population of cells, or individual members thereof, express the molecule. Methods of using a labeled ILP family polypeptide in this manner are known to those of skill in the art. For example, a population of cells could be quickly screened for cells expressing a molecule to which an ILP family polypeptide binds with specificity (e.g., properdin) using a labeled ILP family polypeptide in conjunction with a fluorescence activated cell sorter.

The terms “ILP family protein gene product”, “ILP family protein”, “ILP family peptide” and “ILP family polypeptide” also include biologically functional equivalents and analogs of ILP family proteins. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct ILP family protein analogs. There is no need for an “ILP family protein gene product”, “ILP family protein”, “ILP family peptide” and “ILP family polypeptide” to comprise all or substantially all of the amino acid sequence of a native ILP family protein gene product. Shorter or longer sequences are anticipated to be of use in the presently disclosed subject matter; shorter sequences are herein referred to as “fragments” or “segments”. Thus, the terms “ILP family protein gene product”, “ILP family protein”, “ILP family peptide” and “ILP family polypeptide” also include fragment, fusion, chemically modified, or recombinant ILP family protein polypeptides and proteins comprising sequences of the presently disclosed subject matter. Methods of preparing such proteins are known in the art.

The terms “ILP family protein gene”, “ILP family protein gene sequence”, and “ILP family protein gene fragment” refer to any DNA sequence that is substantially identical to a polynucleotide sequence encoding an ILP family protein gene product, protein or polypeptide as defined above, and can also comprise any combination of associated control sequences. The terms also refer to RNA, or antisense sequences, complementary to such DNA sequences. As used herein, the term “DNA segment” or “DNA fragment” refers to a DNA molecule that has been isolated free of total genomic DNA of a particular species. Furthermore, a DNA segment encoding an ILP family protein refers to a DNA segment that contains ILP family protein coding sequences, yet is isolated away from, or purified free from, total genomic DNA of a source species, such as I. scapularis. Included within the term “DNA segment” are DNA segments and smaller fragments of such segments, and also recombinant vectors, including, for example, plasmids, cosmids, phages, viruses, and the like.

TABLE 1
Novel ILP family proteins and genes encoding same

| Pro- | | | Nucleotide | Amino Acid |
|---|---|---|---|---|
| tein | Name | Clone | SEQ ID NO: | SEQ ID NO: |
| 1 | Salp20-like protein 1 | S20Lclone1 | 1 | 2 |
| 2 | Salp20-like protein 2 | S20Lclone2 | 3 | 4 |
| 3 | Salp20-like protein 3 | S20Lclone3 | 5 | 6 |
| 4 | Salp20-like protein 4 | S20Lclone4 | 7 | 8 |
| 5 | Salp20-like protein 5 | S20Lclone5 | 9 | 10 |
| 6 | Salp20-like protein 6 | S20Lclone6 | 11 | 12 |
| 7 | Salp20-like protein 7 | S20Lclone7 | 13 | 14 |
| 8 | Salp20-like protein 8 | S20Lclone8 | 15 | 16 |
| 9 | Salp20-like protein 9 | S20Lclone9 | 17 | 18 |
| 10 | Salp20-like protein 10 | S20Lclone10 | 19 | 20 |
| 11 | Salp20-like protein 11 | S20Lclone11 | 21 | 22 |
| 12 | Salp20-like protein 12 | S20Lclone12 | 23 | 24 |
| 13 | Salp20-like protein 13 | S20Lclone13 | 25 | 26 |
| 14 | Salp20-like protein 14 | S20Lclone14 | 27 | 28 |
| 15 | Salp20-like protein 15 | S20Lclone15 | 29 | 30 |

The term “substantially identical”, when used to define either an ILP family gene product or amino acid sequence, or a ILP family protein gene or nucleic acid sequence, means that a particular sequence varies from the sequence of a natural ILP family protein or fragment thereof by one or more deletions, substitutions, or additions, the net effect of which is to retain at least some of the biological activity of the natural gene, gene product, or sequence. Such sequences include “mutant” sequences, or sequences in which the biological activity is altered to some degree but retains at least some of the original biological activity.

Alternatively, DNA analog sequences are “substantially identical” to specific DNA sequences disclosed herein if: (a) the DNA analog sequence is derived from coding regions of the natural ILP family protein gene; or (b) the DNA analog sequence is capable of hybridization of DNA sequences of (a) under stringent conditions and which encode biologically active ILP family protein gene product; or (c) the DNA sequences are degenerate as a result of alternative genetic code to the DNA analog sequences defined in (a) and/or (b). Substantially identical analog proteins will be greater than about 80%, or 90% or greater, or about 91%, 92%, 93%, 94%, 95%, 96%, 97%, 98%, or 99% or greater, identical to the corresponding sequence of the native protein or biologically active fragment thereof. Sequences having lesser degrees of identity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids or modifications to amino acids (e.g., chemical modifications) to create biologically functional equivalents.

Sequence identity or percent similarity of a DNA or peptide sequence can be determined, for example, by comparing sequence information using the GAP computer program, available from the University of Wisconsin Geneticist Computer Group. The GAP program utilizes the alignment method of Needleman et al., 1970, as revised by Smith et al., 1981. Briefly, the GAP program defines similarity as the number of aligned symbols (i.e., nucleotides or amino acids) that are similar, divided by the total number of symbols in the shorter of the two sequences. The preferred parameters for the GAP program are the default parameters, which do not impose a penalty for end gaps. See Schwartz et al., 1979; Gribskov et al., 1986.

In certain embodiments, the presently disclosed subject matter concerns the use of ILP family protein genes and gene products that include within their respective sequences a sequence that is essentially that of a ILP family protein gene, or the corresponding protein, or fragments thereof. The term “a sequence essentially as that of a ILP family protein gene”, means that the sequence is substantially identical or substantially similar to a portion of a ILP family protein gene or gene products and contains a minority of bases or amino acids (whether DNA or protein) which are not identical to those of a ILP family protein or a ILP family protein gene, or which are not a biologically functional equivalent. The term “biologically functional equivalent” is well understood in the art and is further defined in detail herein. Nucleotide sequences are “essentially the same” where they have between about 80% and about 85% or in some embodiments, between about 86% and about 90%, or in some embodiments greater than 90%, or in some embodiments about 91%, 92%, 93%, 94%, 95%, 96%, 97%, 98%, or 99%; of nucleic acid residues which are identical to the nucleotide sequence of a ILP family protein gene. Similarly, peptide sequences which have about 80%, or 90% or greater, or about 91%, 92%, 93%, 94%, 95%, 96%, 97%, 98%, or 99% or greater amino acids which are identical or functionally equivalent or biologically functionally equivalent to the amino acids of a ILP family protein polypeptide will be sequences which are “essentially the same”.

ILP family protein gene products and ILP family protein genes encoding nucleic acid sequences, which have functionally equivalent codons, are also covered by the subject matter disclosed herein. The term “functionally equivalent codon” is used herein to refer to codons that encode the same amino acid, such as the ACG and AGU codons for serine. Thus, when referring to the sequence examples presented in SEQ ID NOs: 1-40, for example, the presently disclosed subject matter provides for the substitution of functionally equivalent codons of Table 2 into the sequence examples of SEQ ID NOs: 1-40. Thus, applicants are in possession of amino acid and nucleic acid sequences which include such substitutions but which are not set forth herein in their entirety for convenience.

TABLE 2
Functionally Equivalent Codons

| Amino Acids | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | ACG AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will also be understood by those of ordinary skill in the art that amino acid and nucleic acid sequences can include additional residues, such as additional N- or C-terminal amino acids or 5′ or 3′ nucleic acid sequences, and yet still be essentially as set forth in one of the sequences disclosed herein, so long as the sequence retains biological protein activity where protein expression is concerned. The addition of terminal sequences particularly applies to nucleic acid sequences which can, for example, include various non-coding sequences flanking either of the 5′ or 3′ portions of the coding region or can include various internal sequences, La, introns, which are known to occur within genes.

The present subject matter also encompasses the use of nucleotide segments that are complementary to the sequences of the presently disclosed subject matter, in one embodiment, segments that are fully complementary, i.e. complementary for their entire length. Nucleic acid sequences that are “complementary” are those, which are base-paired according to the standard Watson-Crick complementarity rules. As used herein, the term “complementary sequences” means nucleic acid sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

One technique in the art for assessing complementary sequences and/or isolating complementary nucleotide sequences is hybridization. Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and preferably in excess of about 45° C. Stringent salt conditions will ordinarily be less than about 1,000 mM, typically less than about 500 mM, and preferably less than about 200 mM. However, the combination of parameters is much more important than the measure of any single parameter. See e.g., Wethmur & Davidson, 1968. Determining appropriate hybridization conditions to identify and/or isolate sequences containing high levels of homology is well known in the art. See e.g., Sambrook et al., 2001.

For the purposes of specifying conditions of high stringency, preferred conditions are salt concentration of about 200 mM and temperature of about 45° C. One example of such stringent conditions is hybridization at 4×SSC, at 65° C., followed by a washing in 0.1×SSC at 65° C. for one hour. Another exemplary stringent hybridization scheme uses 50% formamide, 4×SSC at 42° C. Another example of “stringent conditions” refers to conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. Nucleic acids having sequence similarity are detected by hybridization under low stringency conditions, for example, at 50° C. and 10×SSC (0.9 M NaCl/0.09 M sodium citrate) and remain bound when subjected to washing at 55° C. in 1×SSC. Sequence identity can be determined by hybridization under stringent conditions, for example, at 50° C. or higher and 0.1×SSC (9 mM NaCl/0.9 mM sodium citrate).

Nucleic acids that are substantially identical to the provided ILP family protein sequences, e.g., allelic variants, genetically altered versions of the gene, etc., bind to the provided ILP family protein sequences under stringent hybridization conditions. By using probes, particularly labeled probes of DNA sequences, one can isolate homologous or related genes. The source of homologous genes can be any species, e.g., arthropod species, particularly tick species (Order acari), and also including primate species, particularly human; rodents, such as rats and mice, canines, felines, bovines, ovines, equines, yeast, nematodes, etc.

Between arthropod species, e.g., ticks, mites, insects, and spiders, homologs have substantial sequence similarity, i.e. at least about 80%, or 90% or greater, or about 91%, 92%, 93%, 94%, 95%, 96%, 97%, 98%, or 99% or greater sequence identity between nucleotide sequences. Sequence similarity is calculated based on a reference sequence, which can be a subset of a larger sequence, such as a conserved motif, coding region, flanking region, etc. A reference sequence will usually be at least about 18 nucleotides long, more usually at least about 30 nucleotides long, and can extend to the complete sequence that is being compared. Algorithms for sequence analysis are known in the art, such as BLAST, described in Altschul et al., 1990. The sequences provided herein are essential for recognizing ILP family related and homologous proteins in database searches.

At a biological level, identity is just that, i.e. the same amino acid at the same relative position in a given family member of a gene family. Homology and similarity are generally viewed as broader terms. For example, biochemically similar amino acids, for example leucine and isoleucine or glutamate/aspartate, can be present at the same position—these are not identical per se, but are biochemically “similar”. As disclosed herein, these are referred to as conservative differences or conservative substitutions. This differs from a conservative mutation at the DNA level, which changes the nucleotide sequence without making a change in the encoded amino acid, e.g., TCC to TCA, both of which encode serine.

When percentages are referred to herein with regard to polypeptide or polynucleotide homology, it is meant to refer to percent identity. The percent identities referenced herein can be generated, for example, by alignments with the program GENEWORKS™ (Oxford Molecular, Inc. of Campbell, Calif., United States of America) and/or the BLAST program at the NCBI website. Another commonly used alignment program is entitled CLUSTAL W and is described in Thompson et al., 1994, among other places.

Probe sequences can also hybridize specifically to duplex DNA under certain conditions to form triplex or other higher order DNA complexes. The preparation of such probes and suitable hybridization conditions are disclosed herein and are known in the art. By way of example and not limitation, probes used for PCR amplification of Isac, an ILP family protein, are disclosed herein and identified as SEQ ID NOs: 37-40.

The term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

In some embodiments, the presently disclosed subject matter concerns isolated DNA segments and recombinant vectors incorporating DNA sequences, which encode an ILP family protein polypeptide or biologically active fragment thereof that includes within its amino acid sequence an amino acid sequence as described herein. In other particular embodiments, the presently disclosed subject matter concerns recombinant vectors incorporating DNA segments, which encode a protein comprising the amino acid sequence of an ILP family protein (for example, but not limited to SEQ ID NOs: 1-40) or biologically functional equivalents thereof.

III.A. Biologically Functional Equivalents

As mentioned above, modifications and changes can be made in the structure of the ILP family proteins described herein and still constitute a molecule having like or otherwise desirable characteristics. For example, certain amino acids can be substituted for other amino acids or chemically modified (e.g., to increase stability of the peptide) in a protein structure without appreciable loss of interactive capacity with, for example, complement pathway binding proteins or intermediates, including in particular properdin, which can modulate activation and/or activity of the complement pathway. Since it is the interactive capacity and nature of a protein that defines that protein's biological functional activity, certain amino acid sequence modifications or substitutions can be made in a protein sequence (or the nucleic acid sequence encoding it) to obtain a protein with the same, enhanced, or antagonistic properties. Such properties can be achieved by interaction with the normal targets of the native protein, but this need not be the case. It is thus provided in accordance with the present subject matter that various modifications or changes can be made in the sequence of the ILP family proteins and peptides or underlying nucleic acid sequence without appreciable loss of their biological utility or activity.

Biologically functional equivalent peptides, as used herein, are peptides in which certain, but not most or all, of the amino acids can be substituted and/or chemical modifications, substitutions or additions are made to one or more amino acids. Thus, for example, when referring to the sequence examples presented in the even numbered SEQ ID NOs: 2-40, applicants provide for the substitution of codons that encode biologically equivalent amino acids as described herein into the sequence examples of even numbered SEQ ID NOs: 2-40. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged. Changes designed by man can be introduced through the application of site-directed mutagenesis techniques, e.g., to introduce improvements to the antigenicity of the protein or to test ILP family protein mutants in order to examine ILP family protein activity at the molecular level.

Amino acid substitutions, such as those which might be employed in modifying the ILP family proteins and peptides described herein, are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Those of skill in the art will appreciate other biologically functionally equivalent changes.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., 1982, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those, which are within ±1 of the original value, are particularly preferred, and those within ±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those, which are within ±1 of the original value, are particularly preferred, and those within ±0.5 of the original value are even more particularly preferred.

While discussion has focused on functionally equivalent polypeptides arising from amino acid changes, it will be appreciated that these changes can be affected by alteration of the encoding DNA, taking into consideration also that the genetic code is degenerate and that two or more codons can code for the same amino acid.

Thus, it will also be understood that the presently disclosed subject matter is not limited to the particular nucleic acid and amino acid sequences of SEQ ID NOs: 1-40. Recombinant vectors and isolated DNA segments can therefore variously include ILP family polypeptide-encoding regions, include coding regions bearing selected alterations or modifications in the basic coding region, or include larger polypeptides which nevertheless comprise ILP family protein-encoding regions or can encode biologically functional equivalent proteins or peptides which have variant amino acid sequences, or can encode biologically functional equivalent fragments of an entire ILP family protein. Biological activity of an ILP family protein can include binding specificity for properdin and ability to modulate activation and/or activity of the complement pathway. Determining biological activity as described herein is within the ordinary skill of one skilled in the art, upon review of the present disclosure. Exemplary procedures for determining biological activity of ILP family protein polypeptides are disclosed herein in the Examples.

In particular embodiments, the presently disclosed subject matter concerns isolated DNA sequences and recombinant DNA vectors incorporating DNA sequences that encode a protein comprising the amino acid sequence of an ILP family protein. In certain other embodiments, the present subject matter concerns isolated DNA segments and recombinant vectors that comprise a nucleic acid sequence essentially as set forth in the odd numbered SEQ ID NOs: 1-35.

The nucleic acid segments of the present subject matter, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore provided that a nucleic acid fragment of almost any length can be employed, with the total length preferably being limited by the ease of preparation and use in the intended recombinant DNA protocol. For example, nucleic acid fragments can be prepared which include a short stretch complementary, and/or fully complementary, to a nucleic acid sequence set forth in any of the odd numbered SEQ ID NOs: 1-35 such as about 10 nucleotides, and which are up to 10,000 or 5,000 base pairs in length, with segments of 3,000 being preferred in certain embodiments. DNA segments with total lengths of about 4,000, 3,000, 2,000, 1,000, 500, 200, 100, and about 50 base pairs in length are also provided to be useful.

The DNA segments of the present subject matter encompass biologically functionally equivalent ILP family proteins and peptides. Such sequences can arise as a consequence of codon redundancy and functional equivalency that are known to occur naturally within nucleic acid sequences and the proteins thus encoded. Alternatively, functionally equivalent proteins or peptides can be created via the application of chemical synthesis or recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test ILP family protein mutants in order to examine activity in the modulation of, for example, binding specificity for properdin, modulation of complement activity, or other activity at the molecular level. Site-directed mutagenesis techniques are known to those of skill in the art and are disclosed herein.

The presently disclosed subject matter further encompasses fusion proteins and peptides wherein an ILP family protein coding region is aligned within the same expression unit with other proteins or peptides having desired functions, such as for purification, labeling, or immunodetection purposes.

Recombinant vectors form further aspects of the present disclosure. Particularly useful vectors are those in which the coding portion of the DNA segment is positioned under the control of a promoter. The promoter can be that naturally associated with an ILP family protein gene, as can be obtained by isolating the 5′ non-coding sequences located upstream of the coding segment or exon, for example, using recombinant cloning and/or polymerase chain reaction (PCR) technology and/or other methods known in the art, in conjunction with the compositions disclosed herein.

In other embodiments, it is provided that certain advantages will be gained by positioning the coding DNA segment under the control of, i.e. operatively linked to, a recombinant, or heterologous, promoter. As used herein, a recombinant or heterologous promoter is a promoter that is not normally associated with an ILP family protein gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (See, e.g., Sambrook et al., 2001). The promoters employed can be constitutive or inducible and can be used under the appropriate conditions to direct high level expression of the introduced DNA segment, such as is advantageous in the large-scale production of recombinant proteins or peptides. Appropriate promoter systems provide for use in high-level expression include, but are not limited to, the vaccinia virus promoter and the baculovirus promoter.

In an alternative embodiment, the presently disclosed subject matter provides an expression vector comprising a polynucleotide that encodes a biologically active ILP family protein polypeptide in accordance with the present disclosure. In some embodiments, an expression vector of the present subject matter comprises a polynucleotide that encodes an ILP family protein gene product. In another embodiment, an expression vector of the present subject matter comprises a polynucleotide that encodes a polypeptide comprising an amino acid residue sequence of any of evenly numbered SEQ ID NOs: 2-36. In some embodiments, an expression vector of the presently disclosed subject matter comprises a polynucleotide operatively linked to an enhancer-promoter. For example, an expression vector can comprise a polynucleotide operatively linked to a prokaryotic promoter. Alternatively, an expression vector of the presently disclosed subject matter comprises a polynucleotide operatively linked to an enhancer-promoter that is a eukaryotic promoter and the expression vector further comprises a polyadenylation signal that is positioned 3′ of the carboxy-terminal amino acid and within a transcriptional unit of the encoded polypeptide.

In some embodiments, disclosed herein is a recombinant host cell transfected with a polynucleotide that encodes a biologically active ILP family protein in accordance with the present subject matter. SEQ ID NOs: 1-36 set forth representative nucleotide and amino acid sequences of ILP family proteins from ticks. Also provided are homologous or biologically functionally equivalent polynucleotides and ILP family polypeptides found in other animals, including for example other arthropod homologs. Optionally, a recombinant host cell of the present subject matter is transfected with the polynucleotide that encodes a ILP family polypeptide. A recombinant host cell is a bacterial cell, a mammalian cell or an insect cell. In some embodiments, the host cell is an attenuated bacterium, such as for example, attenuated Salmonella and the host is utilized to deliver the ILP family protein polynucleotide sequence to a target cell or tissue within a subject, wherein the ILP family polypeptide is translated from the polynucleotide. Motameni et al., 2004 discloses representative methods for engineering the exemplary attenuated Salmonella host cells, and is incorporated herein by reference in its entirety.

In some embodiments, a recombinant host cell is a prokaryotic host cell, including parasitic and bacterial cells. Preferably, a recombinant host cell is a bacterial cell, for example, a strain of Escherichia coli. The recombinant host cell can comprise a polynucleotide under the transcriptional control of regulatory signals functional in the recombinant host cell, wherein the regulatory signals appropriately control expression of the ILP family polypeptide in a manner to enable all necessary transcriptional and post-transcriptional modification.

In yet another embodiment, provided is a process of preparing an ILP family protein comprising transfecting a cell with polynucleotide that encodes a biologically active ILP family polypeptide as disclosed herein, to produce a transformed host cell, and maintaining the transformed host cell under biological conditions sufficient for expression of the polypeptide. The polypeptide can be isolated if desired, using any suitable technique. The host cell can be a prokaryotic or eukaryotic cell, such as, but not limited to a bacterial cell of Salmonella sp. or Escherichia coli. In some embodiments the host cell can be an insect cell. In some embodiments, a polynucleotide transfected into the transformed cell comprises the nucleotide base sequence of any of the odd numbered SEQ ID NOs: 1-35. SEQ ID NOs: 1-36 set forth nucleotide and amino acid sequences for representative ILP family polypeptides of the presently disclosed subject matter. Also provided are homologs or biologically equivalent ILP family protein polynucleotides and polypeptides found in other vertebrates besides tick species.

As mentioned above, in connection with expression embodiments to prepare recombinant ILP family proteins and peptides, it is provided that longer DNA segments can be used, with DNA segments encoding an entire ILP family protein, biologically active domains or cleavage products thereof, being most preferred. However, it will be appreciated that the use of shorter DNA segments to direct the expression of ILP family protein, epitopes or core regions, such as can be used to generate anti-ILP family protein antibodies, also falls within the scope of the presently disclosed subject matter.

DNA segments which encode peptide antigens from about 5 to about 50 amino acids in length, or more preferably, from about 10 to about 30 amino acids in length can be particularly useful. DNA segments encoding peptides will generally have a minimum coding length in the order of about 15 to about 150, or to about 90 nucleotides. DNA segments encoding full-length proteins can have a minimum coding length on the order of about 500 or 600 nucleotides for a protein in accordance with SEQ ID NOs: 1-36.

III.B. Peptide Modification Techniques and Derivatives

An ILP family protein or biologically functional equivalents thereof of the presently disclosed subject matter can be subject to various changes, substitutions, insertions, and deletions where such changes provide for certain advantages in its use. Thus, the term “polypeptide”, “gene product”, “peptide” and “protein” encompasses any of a variety of forms of peptide derivatives, that include amides, conjugates with proteins, cyclized peptides, polymerized peptides, conservatively substituted variants, analogs, fragments, peptoids, chemically modified peptides, and peptide mimetics. The modifications disclosed herein can also be applied as desired and as appropriate to antibodies.

Additional residues can also be added at either terminus of a peptide for the purpose of providing a “linker” by which the peptides of the presently disclosed subject matter can be conveniently affixed to a label or solid matrix, or carrier. Amino acid residue linkers are usually at least one residue and can be 40 or more residues, more often 1 to 10 residues, but do alone not constitute radiation inducible target ligands. Typical amino acid residues used for linking are tyrosine, cysteine, lysine, glutamic and aspartic acid, or the like. In addition, a peptide can be modified by terminal-NH₂ acylation (e.g., acetylation, or thioglycolic acid amidation) or by terminal-carboxylamidation (e.g., with ammonia, methylamine, and the like terminal modifications). Terminal modifications are useful, as is well known, to reduce susceptibility by proteinase digestion, and therefore serve to prolong half-life of the peptides in solutions, particularly biological fluids where proteases can be present.

Peptides of the presently disclosed subject matter can comprise naturally occurring amino acids, synthetic amino acids, genetically encoded amino acids, non-genetically encoded amino acids, and combinations thereof. Peptides can include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

III.B.1. Peptide Synthesis and Modification

Production of and modifications to the ILP family proteins and peptides described herein can be carried out using techniques known in the art, including site directed mutagenesis and chemical synthesis.

Site-specific mutagenesis is a technique useful in the preparation of individual peptides, or biologically functional equivalent proteins or peptides, through specific mutagenesis of the underlying DNA. The technique further provides a ready ability to prepare and test sequence variants; for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 30 nucleotides in length is preferred, with about 5 to 10 residues on both sides of the junction of the sequence being altered.

In general, the technique of site-specific mutagenesis is well known in the art as exemplified by publications (e.g., Adelman et al., 1983; Sambrook et al., 2001) and can be achieved in a variety of ways generally known to those of skill in the art.

Peptides of the presently disclosed subject matter, including peptoids, can also be chemically synthesized by any of the techniques that are known to those skilled in the art of peptide synthesis. Synthetic chemistry techniques, such as a solid-phase Merrifield-type synthesis, can be used for reasons of purity, antigenic specificity, freedom from undesired side products, ease of production, and the like. A summary of representative techniques can be found in Stewart & Young, 1969; Merrifield, 1969; Fields & Noble, 1990; and Bodanszky, 1993. Solid phase synthesis techniques can be found in Andersson et al., 2000, and in U.S. Pat. Nos. 6,015,561; 6,015,881; 6,031,071; and 4,244,946. Peptide synthesis in solution is described by Schröder & Lübke, 1965. Appropriate protective groups usable in such synthesis are described in the above texts and in McOmie, 1973. In addition, peptides comprising a specified amino acid sequence can be purchased from commercial sources (e.g., Biopeptide Co., LLC of San Diego, Calif., United States of America and PeptidoGenics of Livermore, Calif., United States of America).

III.B.2. Cyclic Peptides

Peptide cyclization is a useful modification because of the stable structures formed by cyclization and in view of the biological activities observed for such cyclic peptides as described herein. An exemplary method for cyclizing peptides is described by Schneider & Eberle, 1993. Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxyl termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

111.8.3. Peptoids

The term “peptoid” as used herein refers to a peptide wherein one or more of the peptide bonds are replaced by pseudopeptide bonds including but not limited to a carba bond (CH₂-CH₂), a depsi bond (CO—O), a hydroxyethylene bond (CHOH—CH₂), a ketomethylene bond (CO—CH₂), a methylene-oxy bond (CH₂—O), a reduced bond (CH₂—NH), a thiomethylene bond (CH₂—S), a thiopeptide bond (CS—NH), and an N-modified bond (—NRCO—). See e.g. Corringer et al., 1993; Garbay-Jaureguiberry et al., 1992; Tung et al., 1992; Urge et al., 1992; Pavone et al., 1993.

III.B.4. Peptide Mimetics

The term “peptide mimetic” as used herein refers to a ligand that mimics the biological activity of a reference peptide, by substantially duplicating the targeting activity of the reference peptide, but it is not a peptide or peptoid. In one embodiment, a peptide mimetic is a small molecule having a molecular weight of less than about 700 daltons.

A peptide mimetic can be designed by: (a) identifying the pharmacophoric groups responsible for the targeting activity of a peptide; (b) determining the spatial arrangements of the pharmacophoric groups in the active conformation of the peptide; and (c) selecting a pharmaceutically acceptable template upon which to mount the pharmacophoric groups in a manner that allows them to retain their spatial arrangement in the active conformation of the peptide. For identification of pharmacophoric groups responsible for targeting activity, mutant variants of the peptide can be prepared and assayed for targeting activity. Alternatively or in addition, the three-dimensional structure of a complex of the peptide and its target molecule can be examined for evidence of interactions, for example the fit of a peptide side chain into a cleft of the target molecule, potential sites for hydrogen bonding, etc. The spatial arrangements of the pharmacophoric groups can be determined by NMR spectroscopy or X-ray diffraction studies. An initial three-dimensional model can be refined by energy minimization and molecular dynamics simulation. A template for modeling can be selected by reference to a template database and will typically allow the mounting of 2-8 pharmacophores. A peptide mimetic is identified wherein addition of the pharmacophoric groups to the template maintains their spatial arrangement as in the peptide.

A peptide mimetic can also be identified by assigning a hashed bitmap structural fingerprint to the peptide based on its chemical structure, and determining the similarity of that fingerprint to that of each compound in a broad chemical database. The fingerprints can be determined using fingerprinting software commercially distributed for that purpose by Daylight Chemical Information Systems, Inc. (Mission Viejo, Calif., United States of America) according to the vendor's instructions. Representative databases include but are not limited to SPREI'95 (InfoChem GmbH of München, Germany), Index Chemicus (ISI of Philadelphia, Pa., United States of America), World Drug Index (Derwent of London, United Kingdom), TSCA93 (United States Environmental Protection Agency), MedChem (Biobyte of Claremont, Calif., United States of America), Maybridge Organic Chemical Catalog (Maybridge of Cornwall, England), Available Chemicals Directory (MDL Information Systems of San Leandro, Calif., United States of America), NCI96 (United States National Cancer Institute), Asinex Catalog of Organic Compounds (Asinex Ltd. of Moscow, Russia), and NP (InterBioScreen Ltd. of Moscow, Russia). A peptide mimetic of a reference peptide is selected as comprising a fingerprint with a similarity (Tanamoto coefficient) of at least 0.85 relative to the fingerprint of the reference peptide. Such peptide mimetics can be tested for binding to a substrate molecule, such as for example properdin using the methods disclosed herein.

Additional techniques for the design and preparation of peptide mimetics can be found in U.S. Pat. Nos. 5,811,392; 5,811,512; 5,578,629; 5,817,879; 5,817,757; and 5,811,515.

III.B.5. Salts of Compositions

Any peptide or peptide mimetic of the presently disclosed subject matter can be used in the form of a pharmaceutically acceptable salt. Suitable acids which are capable of the peptides with the peptides of the presently disclosed subject matter include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Suitable bases capable of forming salts with the peptides of the presently disclosed subject matter include inorganic bases such as sodium hydroxide, ammonium hydroxide, potassium hydroxide and the like; and organic bases such as mono-di- and tri-alkyl and aryl amines (e.g. triethylamine, diisopropyl amine, methyl amine, dimethyl amine and the like), and optionally substituted ethanolamines (e.g. ethanolamine, diethanolamine and the like).

### IV. Introduction of Gene Products

In accordance with the present subject matter, where an ILP family protein gene itself is employed to introduce an ILP family protein gene product, a convenient method of introduction will be through the use of a recombinant vector that incorporates the desired gene, together with its associated control sequences. The preparation of recombinant vectors is well known to those of skill in the art and described in many references, such as, for example, Sambrook et al., 2001, incorporated herein in its entirety.

IV.A. Vector Construction

It is understood that the DNA coding sequences to be expressed, in this case those encoding the ILP family protein gene products, are positioned in a vector adjacent to and operatively linked to a promoter (i.e., under the control of a promoter). It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter.

One can also desire to incorporate into the transcriptional unit of the vector an appropriate polyadenylation site (e.g., 5′-AATAAA-3′), if one was not contained within the original inserted DNA. Typically, these poly-A addition sites are placed about 30 to 2000 nucleotides “downstream” of the coding sequence at a position prior to transcription termination.

While use of the control sequences of the specific gene will be preferred, other control sequences can be employed, so long as they are compatible with the genotype of the cell being treated. Thus, one can mention other useful promoters by way of example, including, e.g., an SV40 early promoter, a long terminal repeat promoter from retrovirus, an actin promoter, a heat shock promoter, a metallothionein promoter, and the like.

As is known in the art, a promoter is a region of a DNA molecule typically within about 100 nucleotide pairs upstream of (i.e., 5′ to) the point at which transcription begins (i.e., a transcription start site). That region typically contains several types of DNA sequence elements that are located in similar relative positions in different genes.

Another type of discrete transcription regulatory sequence element is an enhancer. An enhancer imposes specificity of time, location and expression level on a particular coding region or gene. A major function of an enhancer is to increase the level of transcription of a coding sequence in a cell that contains one or more transcription factors that bind to that enhancer. An enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

An enhancer-promoter used in a vector construct of the present subject matter can be any enhancer-promoter that drives expression in a cell to be transfected. By employing an enhancer-promoter with well-known properties, the level and pattern of gene product expression can be optimized.

For introduction of an ILP family protein gene, a vector construct that will deliver the gene to cells of interest is desired. Viral vectors can be used. These vectors can optionally be a HSV-1, an adenovirus, a retrovirus, such as a Lentivirus, a vaccinia virus vector or an adeno-associated virus; these vectors have been successfully used to deliver desired sequences to cells and tend to have a high infection efficiency. By way of example and not limitation, a suitable vector can be pIBN5-His-TOPO from Invitrogen Corp., Carlsbad, Calif., United States of America. Suitable vector-ILP family protein gene constructs are adapted for administration as pharmaceutically acceptable formulation, as described herein below. Viral promoters can also be of use in vectors of the present subject matter, and are known in the art. By way of example and not limitation, a suitable promoter can be Orgyia pseudotsugata baculovirus promoter, OpIE2.

Commonly used viral promoters for expression vectors are derived from polyoma, cytomegalovirus, Adenovirus 2, and Simian Virus 40 (SV40). The early and late promoters of SV40 virus are particularly useful because both are obtained easily from the virus as a fragment that also contains the SV40 viral origin of replication. Smaller or larger SV40 fragments can also be used, provided there is included the approximately 250 base pair sequence extending from the Hind Ill site toward the Bgl I site located in the viral origin of replication. Further, it is also possible, and often desirable, to utilize promoter or control sequences normally associated with the desired gene sequence, provided such control sequences are compatible with the host cell systems.

The origin of replication can be provided either by construction of the vector to include an exogenous origin, such as can be derived from SV40 or other viral source, or can be provided by the host cell chromosomal replication mechanism. If the vector is integrated into the host cell chromosome, the latter is often sufficient.

Where an ILP family protein gene itself is employed, in some embodiments it can be most convenient to simply use a wild type ILP family protein gene directly. However, it is provided that certain regions of an ILP family protein gene can be employed exclusively without employing an entire wild type ILP family protein gene, including fragments. Optionally, the smallest region needed to modulate biological activity so that one is not introducing unnecessary DNA into cells that receive an ILP family protein gene construct can be employed. The ability of these regions to modulate biological activity can be determined by the assays reported in the Examples.

### V. Methods of Employing the Presently Disclosed Subject Matter

Salp20 was originally identified from a fed nymph cDNA library by probing the library with guinea pig tick immune serum. Once isolated, Salp20 was cloned into an expression vector, which was then transfected into insect cells. Salp20 protein containing C-terminal V5-epitope and 6×-histidine tags was produced and secreted from insect cells into culture media. The protein was then purified from the media and used in functional assays. ILP family proteins, also referred to as Salp20-like (Salp20L) family members, were isolated from a whole fed lymph cDNA and nymphal salivary gland cDNA libraries by using the phage particles as template for PCR with Salp20 and Isac specific primers. Additional family members were expressed and purified following the same procedures as described for Salp20. Recombinant Salp20 and ILP family proteins inhibited the alternative pathway of complement as demonstrated by preventing the lysis of rabbit erythrocytes in the presence of normal human serum. Upon further investigation and as provided herein for the first time, Salp20 and other ILP family proteins dissociated Bb from C3b in the C3 convertases of the alternative complement pathway.

Accordingly, disclosed herein is a family of tick salivary proteins that inhibit the complement pathway by a novel mechanism. In particular, these ILP family proteins inhibit the alternative pathway by inhibiting properdin, a positive regulator of complement activation. By directly interacting with properdin, ILP family proteins cause the dissociation of properdin from the C3 convertase and the subsequent decay acceleration of the convertase. This model is supported by the observations that 1) properdin directly binds to Salp20 and ILP family proteins with a relative affinity that is at least 100 fold higher than the affinity of properdin for C3b; and 2) Salp20 treatment reduces the levels of properdin on preformed C3 convertases and C3bP complexes. The decay accelerating activity of Salp20 and ILP family proteins is unique and distinct from any of the characterized alternative pathway decay accelerating factors.

Additionally, the presently disclosed subject matter demonstrates that Salp20 and ILP family proteins also inhibit the binding of properdin to bacterial surfaces. Properdin can bind to the surface of bacteria, which leads to complement mediated killing of bacteria. Thus, Salp20 and ILP family proteins inhibit complement activation and complement recruitment to bacterial surface.

Properdin is a protein that comprises six type 1 thrombospondin repeats. Thus, the tick proteins, i.e. ILP family proteins, are most likely to bind to type 1 thrombospondin repeats. These repeats are found in many other proteins, some of which are involved in cancer and homeostasis. These repeats are also present in proteins produced by human pathogens. As such, the ILP family proteins disclosed herein can bind to many different proteins with thrombospondin repeats and participate in the regulation of their activity.

Therefore, these tick proteins can be of use for treating human diseases caused by inappropriate complement activation. For example, many diseases caused by inflammation (e.g., arthritis and asthma) are exacerbated by complement activation. Complement activation is also involved in acute injuries, burns, heart disease and some auto immune diseases. Further, in some infectious diseases such as SARS, tissue damage is caused by complement activation.

Further, since these tick proteins have the ability to bind thrombospondin repeats, they are ideal candidates to modulate other proteins having thrombospondin repeats, particularly those associated with chronic illness and disease. It is also envisioned that these proteins can also be used to develop vaccines against ticks.

As such, the presently disclosed subject matter provides isolated and purified biologically active ILP family proteins and nucleic acid molecules encoding same. In some embodiments, the ILP family proteins are capable of specifically binding to properdin and modulating the alternative complement pathway. In some embodiments the ILP family proteins bind to properdin by binding to type 1 thrombospondin repeats. In some embodiments the ILP family proteins bind to any protein or polypeptide with thrombospondin repeats. In some embodiments proteins comprising thrombospondin repeats include proteins involved in cancer, homeostasis and pathogenesis. The presently disclosed subject matter provides methods of employing these unique properties of ILP family proteins as discussed herein.

In some embodiments, the presently disclosed subject matter provides methods and compositions for modulating the activity of proteins with thrombospondin repeats. In some embodiments, an I. scapularis salivary protein can bind to and modulate the activity of a protein with thrombospondin repeats. In some embodiments, the I. scapularis salivary protein is selected from the group including, but not limited to, Isac, Salp20, Salp9, and any ILP family protein, particularly as set forth in any of SEQ ID NOs: 1-36, or combinations thereof. In some embodiments, the proteins with thrombospondin repeats include, but are not limited to, proteins involved in cancer, homeostasis and pathogenesis.

The presently disclosed subject matter provides methods and compositions for suppressing the alternative complement pathway in a subject. In some embodiments, an effective amount of an ILP family protein is administered to a subject in need of suppression of the alternative complement pathway. In some embodiments, the ILP family protein is selected from the group including, but not limited to, Isac, Salp20, Salp9, and any ILP family protein, particularly as set forth in any of SEQ ID NOs: 1-36, or combinations thereof. In some embodiments, the composition comprising an ILP family protein is administered orally. In some embodiments, the composition comprising an ILP family protein is administered intravenously.

The presently disclosed subject matter provides methods and compositions for treating diseases caused by or conditions associated with inappropriate complement activation in a subject. In some embodiments, an effective amount of an ILP family protein is administered to a subject suffering from a disease caused by inappropriate complement activation and/or associated complications and/or at risk for suffering complications associated with a disease caused by inappropriate complement. In some embodiments, an ILP family protein is selected from the group including, but not limited to, Isac, Salp20, Salp9, and any ILP family protein, particularly as set forth in any of SEQ ID NOs: 1-36, or combinations thereof. In some embodiments, the composition comprising an ILP family protein is administered orally. In some embodiments, the composition comprising an ILP family protein is administered intravenously.

The presently disclosed subject matter provides methods and compositions for treating diseases caused by or conditions associated with bacteria mediated inappropriate complement activation in a subject. In some embodiments, an effective amount of an ILP family protein is administered to a subject suffering from a disease caused by inappropriate complement activation and/or associated complications and/or at risk for suffering complications associated with complement activation and complement recruitment to bacterial surfaces. In some embodiments, an ILP family protein is selected from the group including, but not limited to, Isac, Salp20, Salp9, and any ILP family protein, particularly as set forth in any of SEQ ID NOs: 1-36, or combinations thereof. In some embodiments, the composition comprising an ILP family protein is administered orally. In some embodiments, the composition comprising an ILP family protein is administered intravenously.

The presently disclosed subject matter provides methods and compositions for generating an anti-tick vaccine. In some embodiments, the anti-tick vaccine is generated using an ILP family protein. In some embodiments, an ILP family protein is selected from the group including, but not limited to, Isac, Salp20, Salp9, and any ILP family protein, particularly as set forth in any of SEQ ID NOs: 1-36, or combinations thereof.

In some embodiments, an ILP family protein can be used to screen for compounds or molecules that bind to, inhibit, degrade, block or otherwise modify the biological activity of the ILP family protein. In some embodiments, the identification of a compound or molecule capable of modify the biological activity of an ILP family protein can block an ILP family protein secreted in tick saliva from inhibiting the complement pathway. In some embodiments, such a compound or molecule can be administered to a subject to thereby prevent a feeding tick on the subject from going undetected by the subject's immune system.

V.A. Subjects

Further with respect to the therapeutic methods of the presently disclosed subject matter, a representative subject is a vertebrate subject. A representative vertebrate is warm-blooded; a representative warm-blooded vertebrate is a mammal. A representative mammal is a human. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economical importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like.

V.B. Formulations

A composition as described herein optionally comprises a composition that includes a carrier. In some embodiments, particularly with regard to the therapeutic methods, the carrier is a pharmaceutically acceptable carrier in mammals, e.g. humans. Suitable formulations include aqueous and non-aqueous sterile injection-solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The composition can be formulated according to the mode of administration, which can include, but is not limited to systemic administration, parenteral administration (including intravascular, intramuscular, and intraarterial administration), oral delivery, buccal delivery, subcutaneous administration, inhalation, intratracheal installation, surgical implantation, transdermal delivery, local injection, and hyper-velocity injection/bombardment or combinations thereof of administration modes.

The compositions used in the methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art. For example, an ILP family polypeptide, including biologically active fragments and modified polypeptides, and polypeptide mimetics, can be formulated in combination with hydrochlorothiazide, and as a pH stabilized core having an enteric or delayed release coating which protects the active agents until reaching desired regions of the gastrointestinal tract.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

V.C. Doses

The term “effective amount” is used herein to refer to an amount of a composition (e.g., a composition comprising an ILP family protein) sufficient to produce a measurable biological response (e.g., a measurable inhibition of complement activation and/or activity). Actual dosage levels of active ingredients in a composition of the presently disclosed subject matter can be varied so as to administer an amount of the active compound(s) that is effective to achieve the desired response for a particular subject and/or application. The selected dosage level will depend upon a variety of factors including the activity of the composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of an effective dose, as well as evaluation of when and how to make such adjustments, are known to those of ordinary skill in the art of medicine.

For administration of a composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich et al., 1966). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

For oral administration, a satisfactory result can be obtained employing an ILP family polypeptide in an amount ranging from about 0.01 mg/kg to about 100 mg/kg and preferably from about 0.1 mg/kg to about 30 mg/kg. A preferred oral dosage form, such as tablets or capsules, will contain an ILP family polypeptide in an amount ranging from about 0.1 to about 500 mg, preferably from about 2 to about 50 mg, and more preferably from about 10 to about 25 mg.

For parenteral administration, an ILP family polypeptide can be employed in an amount ranging from about 0.005 mg/kg to about 100 mg/kg, preferably about 10 to 50 or 10 to 70 mg/kg, and more preferably from about 10 mg/kg to about 30 mg/kg.

For additional guidance regarding formulation and dose, see U.S. Pat. Nos. 5,326,902; 5,234,933; PCT International Publication No. WO 93/25521; Berkow et al., 1997; Goodman et al., 1996; Ebadi, 1998; Katzung, 2001; Remington et al., 1975; Speight et al., 1997; and Duch et al., 1998.

V.D. Routes of Administration

Suitable methods for administering to a subject an ILP family protein in accordance with the methods of the presently disclosed subject matter include but are not limited to systemic administration, parenteral administration (including intravascular, intramuscular, and intraarterial administration), oral delivery, buccal delivery, subcutaneous administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

The particular mode of administration used in accordance with the methods of the present subject matter depends on various factors, including but not limited to the vector and/or carrier employed, the severity of the condition to be treated, and mechanisms for metabolism or removal of the drug following administration.

### VI. Screening Methods

As disclosed herein, ILP family proteins bind properdin. Properdin acts as a positive regulator of the complement pathway by binding to and stabilizing the C3 convertase. The binding of properdin by an ILP family protein results in the destabilization of C3 convertase, thereby accelerating the decay of the C3 convertase and decreasing the activity of the complement pathway. The presently disclosed subject matter provides numerous ILP family proteins with the ability to bind to properdin and modulate the complement pathway. However, in some embodiments, methods of screening for additional compounds with the ability to bind to properdin or any protein with thrombospondin repeats is also provided.

A method of screening candidate substances for an ability to bind to properdin and modulate the complement pathway is provided in accordance with the presently disclosed subject matter. In some embodiments, the method comprises (a) establishing a test sample comprising properdin; (b) administering a candidate substance or a sample suspected of containing a candidate substance to the test sample; and (c) measuring the binding affinity of the candidate substance to properdin. In some embodiments, the measuring can comprise determining the ability of the candidate substance to modulate the activity of the alternative complement pathway. Further, in some embodiments, the measuring can comprise determining whether or not the candidate substance can block binding of properdin to C3 convertase. In some embodiments, the measuring can comprise determining whether or not the candidate substance can bind to a protein having thrombospondin repeats. In some embodiments, the measuring can comprise determining the competition between a candidate substance and an ILP family protein for binding to properdin.

The test sample can further comprise an indicator. The term “indicator” is meant to refer to a chemical species or compound that is readily detectable using a standard detection technique, such as dark versus light detection, fluorescence or chemiluminescence spectrophotometry, scintillation spectroscopy, chromatography, liquid chromatography/mass spectroscopy (LC/MS), colorimetry, and the like. Representative indicator compounds thus include, but are not limited to, fluorogenic or fluorescent compounds, chemiluminescent compounds, colorimetric compounds, UVNIS absorbing compounds, radionucleotides and combinations thereof.

The ability of the candidate substance to modulate the activity of the complement pathway can determined in any suitable manner. For example, the ability of the candidate substance to modulate activity of the complement pathway can determined by: (i) detecting a signal produced by the indicator upon an effect of the candidate substance on binding properdin to the C3 convertase; and (ii) identifying the candidate substance as a modulator of the activity of the complement pathway based upon an amount of signal produced as compared to a control sample.

In some embodiments, a fluorescence based screening methodology is utilized to identify compositions that can bind with specificity to properdin or a protein having thrombospondin repeats based upon a competitive assay. The method is readily amenable to both robotic and very high throughput systems.

Thus, in one embodiment, a screening method of the present subject matter pertains to a method for a identifying a candidate substance for an ability to modulate activation and/or activity of the complement pathway by binding properdin. The method comprises establishing a test sample comprising properdin and a candidate substance, administering to the test sample an ILP family protein comprising an indicator, incubating the sample for a sufficient time to allow interaction of the ILP family protein and the candidate substance with properdin; and detecting a signal produced by the indicator; and identifying the candidate substance as having an ability to modulate activation and/or the activity of the complement pathway based upon an amount of signal produced by the indicator as compared to a control sample, which did not contain the candidate substance. In the presence of a candidate substance capable of binding properdin at the same region as an ILP family protein, the candidate substance will compete for binding of properdin with an ILP family protein. The greater the affinity of the candidate substance for properdin at the region where the ILP family protein binds, the lower the amount of signal produced and the more promising the candidate substance as a modulator of the complement pathway.

In some embodiments, the candidate substance is a polypeptide, and in some embodiments, the polypeptide is an antibody or functional equivalent fragment thereof. Functional fragments of antibodies are described in detail herein. In some embodiments, a nucleic acid molecule encoding the candidate polypeptide is isolated and purified. Alternatively, in some embodiments, the candidate substance is a small molecule, such as a peptide mimetic of an ILP family protein. Peptide mimetics are described in detail elsewhere herein.

In another embodiment of the screening method of the presently disclosed subject matter, an ILP family protein or active fragment thereof, and properdin can be used for screening libraries of compounds in any of a variety of high throughput drug screening techniques. The components employed in such screening can be free in solution, affixed to a solid support, borne on a cell surface, or located intracellularly. For example, in some embodiments, properdin, or a protein having thrombospondin repeats, is immobilized to a solid support and the ILP family polypeptides and candidate substances are allowed to compete for binding to the immobilized properdin. The solid support can then be easily washed to remove unbound substances. The formation of binding complexes, between the ILP family polypeptide or candidate substance with the properdin, can then be measured as described herein. By way of example and not limitation, the formation of binding complexes can be determined by analyzing a phage display of the screened library of candidate compounds.

### Examples

The following Examples provide illustrative embodiments. In light of the present disclosure and the general level of skill in the art, those of ordinary skill will appreciate that the following Examples are intended to be exemplary only and that numerous changes, modifications, and alterations can be employed without departing from the scope of the presently claimed subject matter.

### Materials and Methods for Example 1

Ticks and Tick Saliva

Ixodes scapularis ticks were raised as previously described by Sonenshine (1993). Tick saliva was produced following a modified protocol from Tatchell (1967). Briefly, adult ticks were allowed to feed on New Zealand white rabbits for 5 days. The ticks were removed and attached to glass slides with adhesive tape. Capillaries were placed over the mouthparts, and ˜1-2 μl of pilocarpine (25 mg/ml) and dopamine (25 mg/ml) in 95% ethanol were applied on the dorsum of the ticks. The ticks were allowed to salivate into the capillaries ˜2 h at 27° C. in humidity chambers.

Cell Lines and Media

Adherent cultures of High Five cells (Invitrogen Corp., Carlsbad, Calif., United States of America), derived from the cabbage looper, Trichoplusia ni, were seeded and maintained according to the instructions of the manufacturer. The cells were grown in GIBCO® Express Five Serum free media (SFM) (Invitrogen Corp., Carlsbad, Calif., United States of America) supplemented with L-glutamine (18 mM), penicillin (100 U/ml), streptomycin (100 μg/ml), and GIBCO® fungizone (0.25 μg/ml) (Invitrogen Corp., Carlsbad, Calif., United States of America) at 28° C.

Borrelia burgdorferi B31C1 and Borrelia garinii (ATCC®, Manassas, Va., United States of America) were grown and maintained in complete BSK-II media at 33° C. as described by Ohnishi et al. (2001).

PCR from Ixodes scapularis bacteriophage libraries

In order to construct the cDNA library from 48 h fed nymphs, total RNA was first extracted from ˜300 fed Ixodes scapularis nymphs using the ToTALLY RNA™ extraction kit (Ambion, Inc., Austin, Tex., United States of America) according to the instructions of the manufacturer. Isolation and purification of mRNA from total RNA was performed using the POLY(A)PURIST™ mRNA Purification Kit (Ambion, Inc., Austin, Tex., United States of America). The cDNA library from 48 h fed nymphal ticks was subsequently constructed in the phagemid vector, pBK-CMV, using the ZAP Express® cDNA Synthesis and ZAP Express® cDNA Gigapack III Gold Cloning Kit (Stratagene Corp., La Jolla, Calif., United States of America). The average size of an insert in the phagemid vector was approximately 1.8 kilobases (kb). The titre of the resulting phage library was 2.0×10⁹ plaque forming units (pfu)/ml with a complexity of 1.0×10⁶ clones. The method used to generate the cDNA library generated from fed nymph salivary glands has been described previously (Das et al., 2001).

Products were PCR amplified from each of the bacteriophage libraries directly using the following primer sets: KS20F-5′-CCAGCCATGAGGACTGCGCT-3′ (SEQ ID NO: 37), S20R-5′-TCAGGAAATTGCCTCGAATTGAGT-3′ (SEQ ID NO: 38), IsacF-5′-CACTGAGGTTCAGAGCAAG-3′ (SEQ ID NO: 39), and IsacR-5′-GTATCAGAACTGTGCTTGCAC-3′ (SEQ ID NO: 40). The Salp20 primers, KS20F and S20R, anneal to the 5′ and 3′ ends of the salp20 ORF, while the Isac primers, IsacF and IsacR, anneal upstream and downstream of the isac ORF, respectively. After amplification, the PCR products were cloned into pCR2.1 TOPO® following the instructions of the manufacturer (Invitrogen Corp., Carlsbad, Calif., United States of America). Plasmids containing the PCR products were purified using the QIAprep® Mini-Prep Kit (Qiagen, Inc., Valencia, Calif., United States of America) and then transformed into chemically competent Escherichia coli TOP 10 cells. Transformants were selected and screened by restriction digests of plasmid DNA and PCR analysis using M13F and M13R primers (Invitrogen Corp., Carlsbad, Calif., United States of America), which anneal outside of the multiple cloning region of pCR2.1 TOPO. To determine the identity of the PCR products, plasmids containing inserts of the correct size were sequenced using the M13 primers at the University of North Carolina, Chapel Hill Genome Analysis Facility.

### Example 1

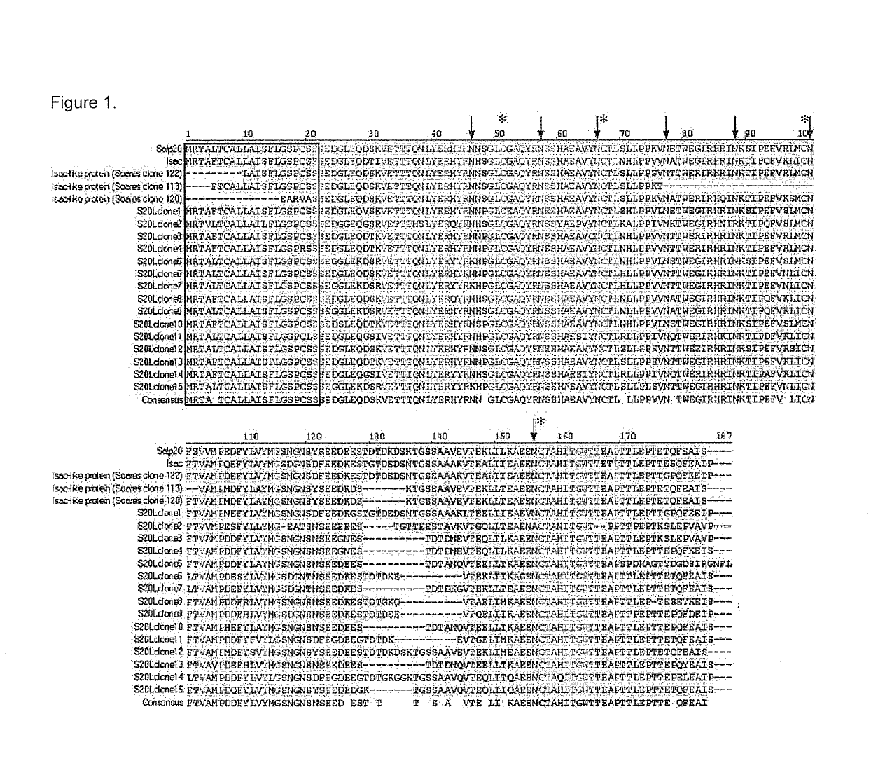

Disclosed herein are 15 unique clones related to Isac and Salp20, increasing the size of the Isac protein family. In order to identify members of this family, products were PCR amplified using two primer sets, S20F & S20R and IsacF & Isac R, from two different cDNA libraries, one generated from the salivary glands of fed I. scapularis nymphs and the other from whole fed I. scapularis nymphs. The PCR products were sequenced and 15 unique clones sharing homology with Isac and Salp20 were identified (FIG. 1). The translated amino acid sequences of each of the isolated clones ranged from 69 to 95% sequence similarity to Isac and Salp20. Twelve of the 15 unique clones contained a 5-10 amino acid deletion at positions 134 through 146 (FIG. 1). Additionally, S20Lclone 5 contained a frameshift mutation at position 171 altering the location of the stop codon by 3 amino acids. In all of the clones identified, a putative secretion signal was present, four cysteines in the mature protein were conserved, and four of the seven N-linked glycosylation sites found in Isac and Salp20 were maintained (FIG. 1).

In FIG. 1, clones identified by PCR analysis from whole tick and salivary gland cDNA libraries were aligned with Salp20, Isac, and cDNA clones previously identified by Soares et al. (2005). Boxed light grey residues indicate conserved amino acids in all clones, and dark grey boxed residues indicate conservation among some of the clones. The putative secretion signals of Salp20, Isac, and all cDNA clones are boxed. Potential N-linked glycosylation sites are marked by arrows and cysteines conserved in the mature proteins of all clones are indicated by asterisks.

### Discussion of Example 1

Provided in the presently disclosed subject matter are additional members of the ILP family. All family members identified except Salp9 possess putative secretion signals, contain 4 conserved cysteines in the mature protein, and retain at least four of the seven N-linked glycosylation sites. An individual tick can express many or all of these genes, with expression patterns that change over time or in different tissues. Alternatively, proteins encoded by genes of this family with potentially similar functions within an individual tick can display antigenic variation, which could be needed to escape host immune responses during prolonged feeding periods. However, since the family members were isolated from cDNA libraries generated from hundreds of nymphal ticks, variation in the sequences might be a result of genetic variation between individual ticks, rather than each tick possessing several family members.

### Materials and Methods for Examples 2-5

Recombinant Proteins, Purified Proteins, and Antibodies

Salp20 and chloramphenicol acetyl-transferase proteins containing C-terminal V5-epitope and 6×-histidine (His) tags (S20NS and CAT, respectively) were expressed and purified from stably transfected High Five cells as described previously (Tyson et al., 2007). Recombinant protein purity was determined by SDS-PAGE, while purified protein concentrations were determined by Bradford analysis. Purified human complement components, C3b, fB, fD, and properdin, and antibodies directed against the complement components, goat α-human C3, goat α-human fB, and goat α-human properdin, were obtained from CompTech (Complement Technology, Inc., Tyler, Tex., United States of America). Mouse α-His IgG was obtained from Qiagen, Inc., Valencia, Calif., United States of America, and mouse α-V5 IgG was obtained from Invitrogen Corp., Carlsbad, Calif., United States of America.

Assays to Measure the Decay of C3 Convertases

To measure the decay of C3 convertases formed from complement components in NHS in the presence of S20NS, enzyme-linked immunosorbent assasys (ELISAs) were performed as described previously (Valenzuela et al., 2000; Tyson et al., 2007). Briefly, microtiter plates were coated with 0.1% agarose for 48 h at 37° C. To form C3 convertases in the wells, the agarose coated wells were then incubated with NHS in AP Buffer (gelatin veronal buffer with Mg²⁺ and Ca²⁺ (GVB⁺⁺, CompTech), 5 mM EGTA, 5 mM MgCl₂) for 1 h at 37° C. The plate bound convertases were subsequently washed and incubated with various concentrations of S20NS for 30 min at 37° C. After incubation, the wells were washed with wash buffer (TBS, 10 mg/ml BSA, 2 mM MgCl₂), and any remaining plate-bound Bb or properdin were detected by standard ELISA methods using either a primary goat α-human fB Ab or a goat α-human properdin Ab, followed by a secondary alkaline phosphatase (AP)-conjugated rabbit a-goat IgG. OD₄₀₅ values were determined and percent deposition was calculated using the following equation: ((OD₄₀₅ sample−OD₄₀₅ NHS with 25 mM EDTA)/(OD₄₀₅ sample without S20NS or CAT−OD₄₀₅ NHS with 25 mM EDTA))×100.

To measure the decay of C3 convertases formed from purified components in the presence of S20NS, an ELISA adapted from Hourcade et al. (2006) was performed. Microtiter plate wells were coated with 250 ng/well of C3b in PBS for 12 h at 4° C. After coating, the wells were washed with PBS and then blocked for 15 min at 23° C. with binding buffer (PBS, 75 mM NaCl , 5 mM NiCl₂, 4% BSA, 0.05% TWEEN®-20). To form the C3 convertase, fB (400 ng/well) and fD (25 ng/well) in Binding buffer were added to the wells and incubated at 37° C. for 2 h. The wells were subsequently washed with PBS and then incubated with various concentrations of S20NS, CAT, or fH in binding buffer for 30 min at 37° C. The wells were washed with TBST (TBS, 0.2% TWEEN®-20), and the OD₄₀₅ was determined for any remaining Bb by ELISA using specific antibodies. Percent deposition was calculated using the following equation: ((OD₄₀₅ sample−OD₄₀₅ C3b coated wells)/(OD₄₀₅ sample without S20NS or CAT−OD₄₀₅ C3b coated wells))×100.

In some assays, properdin was included in the formation of the C3 convertase from purified complement components. After coating the wells with C3b, fB (50 ng/well), fD (25 ng/well), and properdin (50 ng/well) in Mg2+ binding buffer (PBS, 75 mM NaCl, 10 mM MgCl₂, 4% BSA, 0.05% TWEEN®-20) were incubated in the wells for 2 h at 37° C. Plate bound Bb and properdin were detected by standard ELISAs. In these assays, the concentration of fB was lower than in the assays lacking properdin because properdin stabilized the C3 convertase more efficiently than the substitution of Mg²⁺ with Ni²⁺ in the assays lacking properdin. Since the convertase was stabilized more efficiently, less fB was needed to achieve equivalent OD₄₀₅ readings for fB deposition between the two assays. Percent deposition was calculated as described above. To form C3bP complexes, plates were coated with C3b as described above and properdin (50 ng/well) was subsequently added. Bound properdin was detected as described.

Cofactor Activity Assays

To investigate the cofactor activity of S20NS during fI mediated degradation of C3b, cofactor activity assays were performed following a modified protocol of McRae et al. (2005). Briefly, 200 ng of C3b was incubated with various concentrations of S20NS, fH, or CAT and 400 ng of fI in reaction buffer (10 mM Tris-Cl pH 7.5, 150 mM NaCl) for 30 min at 37° C. After incubation, C3b degradation products were analyzed by immunoblots using a primary goat α-C3 Ab and a secondary AP-conjugated rabbit α-goat IgG.

To determine if S20NS degraded C3b in the presence of fH, 200 ng of C3b were incubated with 400 ng of either S20NS or fI and 1 μg of fH in reaction buffer for 30 min at 37° C. C3b degradation products were then detected by immunoblotting.

Assays to Detect Salp20 Binding to Properdin

To detect direct binding of S20NS to properdin, we performed immunoprecipitations and analyzed the precipitates by immunoblot. S20NS (150 ng) was incubated with properdin (450 ng) at 37° C. for 30 min in binding buffer (PBS, 75 mM NaCl, 10 mM MgCl2, 0.05% TWEEN®-20) and then added to blocked Protein-A SEPHADEX® beads (Sigma Aldrich, Corp., St. Louis, Mo., United States of America) coated with 1 μg of mouse α-V5 IgG for 1 hour at 37° C. The SEPHADEX® beads were washed and resuspended in non-reducing SDS-PAGE loading dye. Samples were subjected to SDS-PAGE and immunoblotting with antibodies specific for either S20NS or properdin.

As an alternative method to detect S20NS binding to properdin, microtiter plate wells were first coated with 100 ng/well of S20NS, CAT, or C3b for 12 h at 4° C. The wells were then blocked and incubated with 100 ng/well properdin for 1 h at 37° C. After incubation, the wells were washed. To detect plate bound properdin, the wells were incubated with a primary goat α-properdin Ab and a secondary AP-conjugated rabbit α-goat IgG.

Saturation Binding Assays

To determine the relative binding affinity of properdin for either S20NS or C3b, a solid-phase binding assay was performed. Microtiter plates were coated with a saturating amount of either S20NS (10 ng/well) or C3b (10 ng/well) for 12 hrs at 4° C. in 0.1M Carbonate Binding Buffer, pH 9.2. After coating, the wells were blocked with binding buffer for 1 hr at 37° C. and then incubated with increasing concentrations of properdin in binding buffer (PBS, 75 mM NaCl, 10 mM MgCl₂, 0.05% TWEEN®-20) at 37° C. for 1 hr. The wells were then washed with TBST, and bound properdin was detected by an ELISA using a primary goat α-human properdin Ab and a secondary AP-conjugated rabbit α-goat Ab. Development of the substrate was stopped after 3 min by the addition of 3M NaOH. The OD₄₀₅ was determined and plotted, and relative K_{d} values were calculated using GRAPHPAD PRISM 4® (GraphPad Software, Inc., La Jolla, Calif., United States of America).

### Example 2

S20NS Specifically Inhibits the Alternative Complement Pathway by Dissociating the C3 Convertase

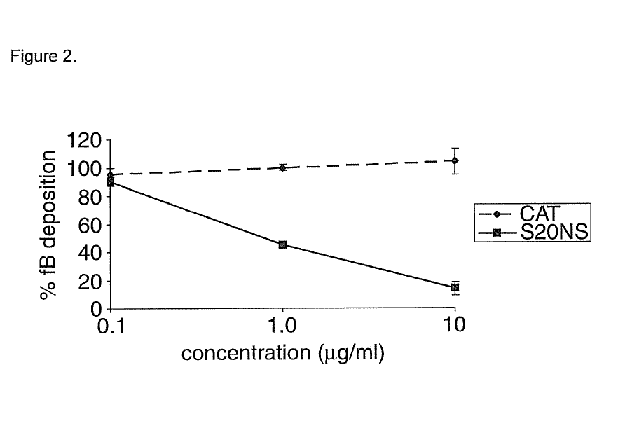

The mechanism of inhibition of the alternative pathway by S20NS was elucidated by performing an agarose based ELISA as described previously (Valenzuela et al., 2000). In this assay, C3 present in NHS is activated by agarose coated microtiter plates. C3 activation leads to the formation of an active convertase on the agarose comprising covalently bound C3b and Bb (Valenzuela et al., 2000). When increasing concentrations of S20NS were incubated with preformed covalently bound C3 convertases, the amount of bound Bb was reduced (IC₅₀ of S20NS=0.8 μg/ml) (FIG. 2). Equal concentrations of purified recombinant CAT protein, a negative control protein expressed from the same expression vector as S20NS in High Five cells, did not disrupt the C3 convertase. Previous studies have demonstrated that covalently attached C3b is unaffected in the presence of S20NS (Tyson et al., 2007). These results indicate that S20NS inhibits the alternative complement pathway by specifically dissociating Bb from the C3 convertase. Since the IC₅₀ of S20NS=0.8 μg/ml, concentrations of either 1 or 2 μg/ml of S20NS were chosen for subsequent experiments.

In FIG. 2, the C3 convertases were preformed on agarose surfaces from complement components in NHS. Ten-fold dilutions of S20NS or CAT were then added to the preformed convertases and the amount of remaining Bb was determined by ELISA. The error bars represent 2 standard deviations from the mean where N=6.

### Example 3

S20NS is a Unique Regulator of the Alternative Pathway

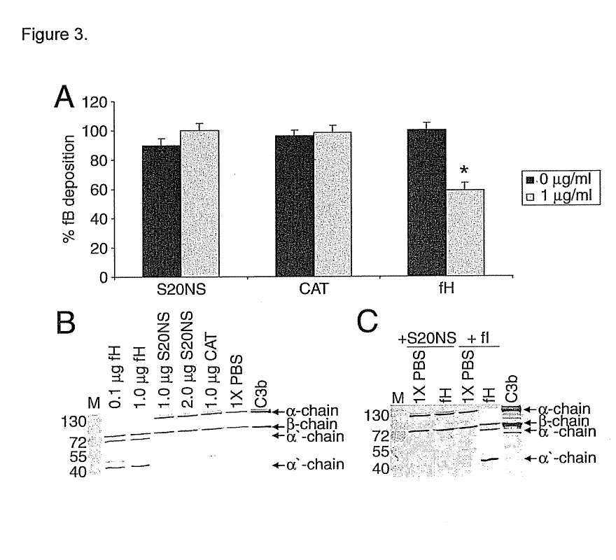

Since S20NS and Isac inhibit the alternative pathway by dissociating Bb from the C3 convertase, it has been hypothesized that Salp20 and Isac act in a manner similar to fH, a natural negative regulator of the alternative pathway. Human fH is a serum glycoprotein that directly binds C3b, displacing Bb and causing decay acceleration of the C3 convertase (Zipfel et al., 1999; Weiler et al., 1976). In addition, fH also acts as a cofactor for fI mediated degradation of C3b (Zipfel et al., 1999; Pangburn et al., 1977; Ross et al., 1982). To determine if S20NS acted by the same mechanism as fH, ELISAs were performed to measure the decay of C3 convertases in the presence of S20NS or fH. In these assays, C3 convertases were formed in the wells of microtiter plates from purified complement components (C3b, fB, and fD) and then incubated S20NS or various control proteins with the convertases. After the incubation, any remaining bound Bb in the convertases was detected by ELISA. The C3 convertases formed from purified components were disrupted by fH as indicated by the reduction in the amount of deposited Bb (FIG. 3A). Surprisingly, however, S20NS displayed no effect (FIG. 2A). These results indicate that in this assay S20NS does not share similar activity to fH. Moreover, these results also demonstrate that S20NS dissociates C3 convertases formed from NHS but not convertases formed from purified complement components (FIG. 3A).

In FIG. 3A, C3 convertases were preformed in microtiter plate wells from purified complement components (C3b, fB, and fD) and washed. S20NS (1 μg/ml), fH (1 μg/ml), CAT (1 μg/ml, negative control), or buffer alone (0 μg/ml) were then added to the preformed convertases and the amount of remaining bound Bb was determined by ELISA. The error bars represent 2 standard deviations from the mean where N=6. The asterisk indicates statistical significance (p=0.008) between the 0 μg/ml and 1 μg/ml samples of fH as measured by a student t-test.

Experiments were also performed to determine if S20NS acted as a cofactor for fI mediated degradation of C3b, similar to fH. S20NS was mixed with purified fI and the mixture was then added to purified C3b. Degradation products of the C3b α-chain (C3b α′-chain), 67 and 43 kDa fragments, were detected by immunoblots with specific Abs. Various concentrations of S20NS or fH were incubated with C3b in the presence of fI, and C3b degradation products, represented by the 67 and 43 kDa bands, were visualized by Western blots using a polyclonal goat α-hC3 Ab. As illustrated in FIG. 3B, various concentrations of either S20NS or CAT were incapable of mediating fI degradation of C3b, unlike fH, which when incubated in the presence of fI, resulted in the degradation of C3b.

Since S20NS did not act as a cofactor for fI mediated C3b degradation like fH, experiments were done to test if S20NS functioned similarly to fI and degraded C3b in the presence of fH. When S20NS was mixed with fH and then incubated with C3b, no degradation of C3b was observed, whereas fI incubated with fH and C3b resulted in C3b degradation (FIG. 3C). C3b degradation products were visualized by Western blots as described in FIG. 2B. In FIG. 3C, M=marker. Together, these results demonstrate that S20NS disrupts the C3 convertase by a mechanism that is different from both fH and fI.

### Example 4

S20NS Inhibits the Alternative Pathway by Displacing Properdin from the C3 Convertase

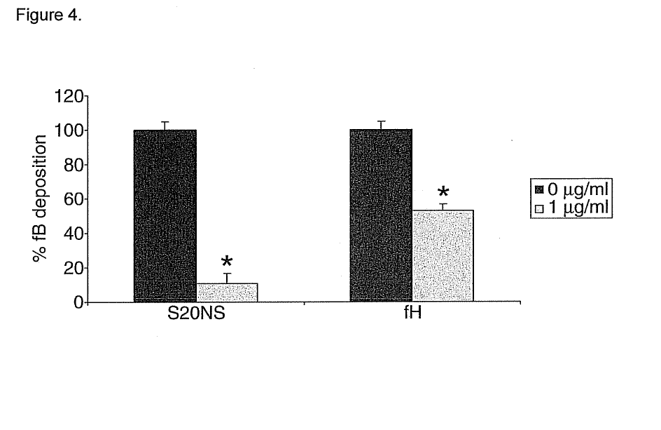

S20NS dissociated the components of the C3 convertase when the convertase was formed from NHS but not from purified complement components (FIG. 3A). The discrepancy in the activity of S20NS between the two assays is likely due to differences in the composition of the convertases formed from either NHS, which potentially contain C3b, Bb, and properdin, or from purified complement components, which contain only C3b and Bb. Properdin is a positive regulator of the alternative pathway that binds and stabilizes the C3 convertase, significantly increasing its half life (Hourcade, 2006; Fearon et al., 1975). To determine if the inhibitory activity of S20NS was potentially mediated through properdin, we formed C3 convertases were formed from purified complement components in the presence of properdin and then incubated S20NS or control proteins with the convertases. When S20NS was incubated with C3 convertases containing properdin, approximately 90% of Bb was displaced (FIG. 4), in contrast to its effect on convertases lacking properdin (FIG. 3A). Factor H displaced Bb from C3 convertases formed in either the presence or absence of properdin (FIG. 3A and FIG. 4).

In FIG. 4, C3 convertases were formed from purified components (C3b, fB, and fD) in the presence of properdin and then washed. S20NS (1 μg/ml), fH (1 μg/ml), or buffer (0 μg/ml) were added to the preformed convertases and the amount of remaining bound Bb was determined by ELISA. The error bars represent 2 standard deviations from the mean where N=6. The asterisks indicate statistical significance between the 0 μg/ml and 1 μg/ml samples as measured by a student's t-test where p<0.001.

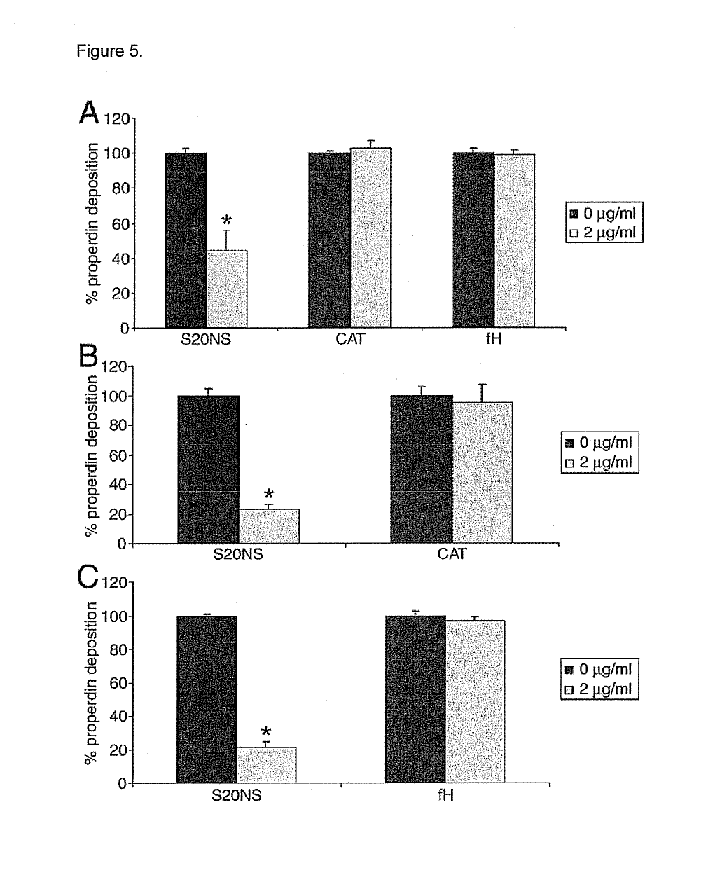

After establishing that S20NS was only active against convertases containing properdin, experiments were done to determine if S20NS displaced properdin from the C3 convertase. In FIG. 5A, C3 convertases were formed from purified complement components as described in FIG. 4. In FIG. 5B, C3 convertases were formed from complement components in NHS as described in FIG. 3. S20NS (2 μg/ml), CAT (2 μg/ml, negative control), or buffer (0 μg/ml) were then incubated with the preformed convertases and bound properdin was detected by ELISA. S20NS (2 μg/ml), fH (2 μg/ml) or buffer (0 μg/ml) were then incubated with the preformed convertases, and bound properdin was detected by ELISA. S20NS displaced properdin from C3 convertases formed from purified components (FIG. 5A) as well as from convertases formed from NHS (FIG. 5B). In addition, S20NS also displaced properdin from complexes containing only C3bP, demonstrating the specificity of S20NS for properdin (FIG. 5C). In FIG. 5C, C3bP complexes were formed from purified C3b and properdin. S20NS (2 μg/ml), fH (2 μg/ml) or buffer (0 μg/ml) were then incubated with the complexes, and bound properdin was detected by ELISA. The error bars represent 2 standard deviations from the mean where N=6. The asterisks indicate statistical significance between the 0 μg/ml and 2 μg/ml samples of S20NS as measured by a student's t-test where p<0.001. Unlike S20NS, fH did not displace properdin from C3 convertases (FIG. 5A) or from C3bP complexes (FIG. 5C). Together, these results demonstrate that S20NS accelerates the decay of C3 convertases by specifically displacing properdin from the convertase.

### Example 5

S20NS Binds Properdin

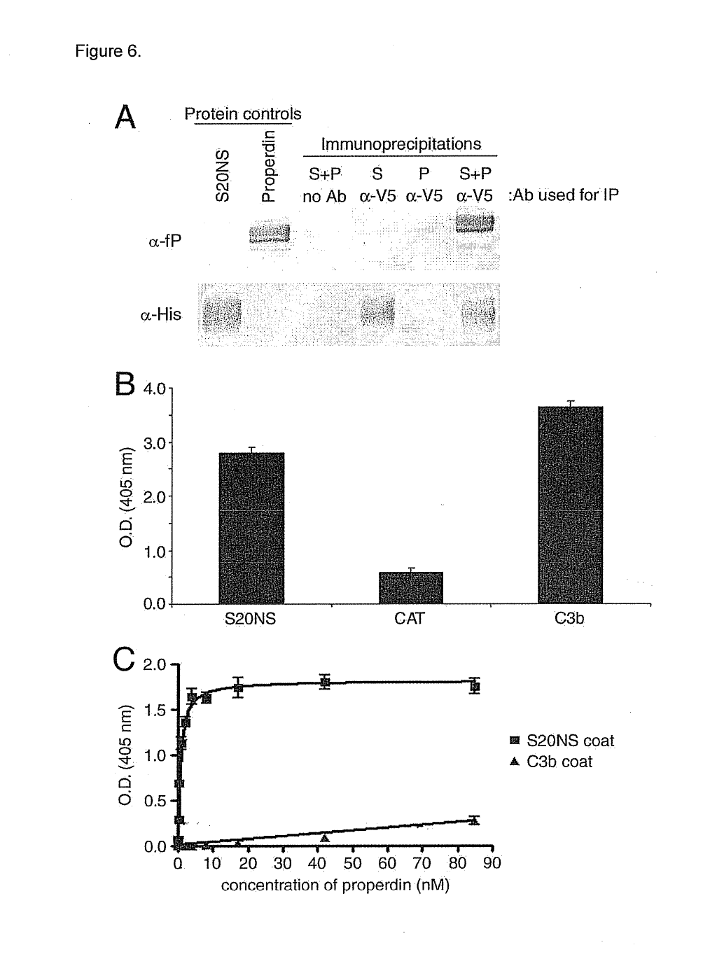

To determine if S20NS directly interacted with properdin to dissociate the C3 convertase, S20NS and properdin were incubated together and S20NS was next immunoprecipitated using an antibody that bound to its C-terminal V5-epitope tag. The precipitates were then immunoblotted for either S20NS or properdin with specific Abs. In the immunoblots, we detected S20NS as well as properdin in the precipitates (FIG. 6A), indicating that S20NS directly bound to properd in.

In FIG. 6A, S20NS (S), properdin (P), or S20NS previously incubated with properdin (S+P) were immunoprecipitated (IP) with a monoclonal α-V5 Ab against an epitope tag on S20NS. Immunoprecipitates were then analyzed by Western Blots using specific Abs directed against properdin (α-fP) or S20NS (α-His).

The interaction between Salp20 and properdin was also confirmed by ELISA. Microtiter plate wells were coated with S20NS and then incubated with properdin. After incubation, bound properdin was detected with specific Abs. In wells coated with either S20NS or C3b, we detected specific binding of properdin when compared to the negative control, CAT (FIG. 6B).

In FIG. 6B, microtiter plate wells were coated with S20NS, CAT (negative control), or C3b (positive control). The wells were washed, blocked, and then incubated with properdin. Bound properdin was detected by ELISA. The error bars represent 2 standard deviations from the mean where N=6.

In addition to studying the direct interaction between S20NS and properdin, the relative binding affinity of properdin for either S20NS or C3b was also calculated by performing solid-phase saturation binding assays. In these assays, microtiter plates were coated with equal amounts of either S20NS or C3b. Increasing concentrations of properdin were then added to the wells, and bound properdin was detected with specific antibodies. Properdin binding to S20NS saturated at a lower concentration than properdin binding to C3b (FIG. 5C). The relative K_{d} of properdin binding to S20NS=0.669 nM where the relative K_{d} of properdin binding to C3b>85 nM. These results indicate properdin binds to S20NS with an affinity that is >100 fold higher than its affinity for C3b.

In FIG. 6C, microtiter plate wells were coated with either S20NS or C3b. Increasing concentrations of properdin were then added to the wells, and bound properdin was detected by ELISA. The data depict a single experiment performed in triplicate that is representative of 3 independent experiments. The error bars represent the standard error from the mean.

### Discussion of Examples 2-5

Examples 2-5 demonstrate that S20NS is only active against C3 convertases containing properdin. While it is not desired to be bound by any particular theory of operation, the simplest mechanism consistent with the data is that S20NS directly interacts with properdin, causing its dissociation from the C3 convertase and the subsequent decay acceleration of the convertase. This model is supported by the observations that 1) properdin directly bound to Salp20 with a relative affinity that was at least 100 fold higher than the affinity of properdin for C3b and 2) Salp20 treatment reduced the levels of properdin on preformed C3 convertases and C3bP complexes. However, alternative models such as properdin facilitating necessary contacts between Salp20 and C3bBb, allowing S20NS to bind the convertase directly and cause decay acceleration, cannot currently be ruled out. However, since no S20NS have been found physically associated with the inactivated convertase, the model in which Salp20 acts by directly displacing properdin from the convertase is favored.

All of the studies were performed with insect cell expressed recombinant S20NS, which are believed to function almost identically to native Salp20 expressed in tick saliva. Valenzuela et al. have demonstrated that native Isac, purified directly from tick salivary gland extracts, inhibited the alternative complement pathway (Valenzuela et al., 2000).

The decay accelerating activity of S20NS is unique and distinct from any of the characterized alternative pathway decay accelerating factors, DAF, CR1, and fH, which directly interact with C3bBb or C3b to destabilize the C3 convertase (Weiler et al., 1976; Morgan et al., 1999; Pangbum et al., 1986; Fujita et al., 1987; Nicholson-Weller et al., 1982; Fearon, 1979; Whaley et al., 1976). S20NS displaced properdin from C3 convertases and C3bP complexes, whereas fH did not displace properdin in our assays. In a previous study, Hourcade used surface plasmon resonance to demonstrate that fH binding to C3 convertases results in the decay of C3 convertases and the dissociation of properdin (Hourcade, 2006). In the instant Examples, properdin dissociation following fH treatment might not have been observed because C3 complexes formed in the ELISAs differ from the convertases formed in the surface plasmon resonance study. Specifically, the C3 convertase complexes formed in the present assays are likely to contain both complete C3 convertases and C3bP complexes. The properdin displaced by S20NS in the present assays might be mainly derived from C3bP complexes, which are not affected by fH.

Even though properdin is not an active component of the C3 convertase, it plays a role in the stabilization and full activity of the convertase (Fearon, 1975; Gupta-Bansal et al., 2000). Gupta-Bansal et al. and Perdikoulis et al. have demonstrated that Abs directed against properdin are capable of inhibiting the alternative pathway (Gupta-Bansal et al., 2000; Perdikoulis et al., 2001). Recent studies have also shown that properdin is capable of binding to cell surfaces and initiating the alternative pathway by providing a platform for the assembly of the C3 convertase (Spitzer et al., 2007). Since properdin plays a role in effective complement activation, it is an attractive target for inactivation by pathogens or blood feeding organisms. One example of a virulence factor that targets properdin is streptococcal pyrogenic exotoxin B, which acts to degrade properdin, allowing the pathogenic group A streptococci to resist opsonophagocytosis mediated by complement (Tsao et al., 2006).

Salp20 is a member of the ILP family, containing at least 49 members (Soares et al., 2005; Ribeiro et al., 2006; Tyson et al., 2007; Daix et al., 2007). There are several other members of this family, for example, Isac, Irac I, Irac II, S20Lclone 12, and S20Lclone 2 (Valenzuela et al., 2000; Tyson et al., 2007; Daix et al., 2007). Based on the present disclosure it is believed that these proteins also interact with properdin.

Properdin is composed of short N- and C-terminal regions separated by 6 TSRs (Goundis et al., 1988), which make up the majority of the protein. While it is not desired to be bound by any particular theory of operation, it is proposed that Salp20 and other ILP family members specifically bind the TSRs of properdin to cause its displacement from the C3 convertase. The TSRs found in properdin and several other proteins primarily bind sulfated glycoconjugates and glycosaminoglycans (GAGs) (Holt et al., 1990; Guo et al., 1992). Interestingly, S20NS contains multiple N- and O-linked glycans that make up almost half the molecular weight of the mature protein. These carbohydrate modifications could potentially be sulfated glycoconjugates and GAGs, allowing S20NS to resemble the sulfated glycoconjugates and bind the TSRs of properd in.

In addition to properdin, TSRs are found in other complement proteins, cell adhesion molecules, and proteases, many of which regulate host hemostasis and innate immunity (Tucker, 2004). In addition to their roles in complement inhibition, ILP family members can target different TSR containing proteins to alter host hemostasis and innate immunity, facilitating tick feeding.

### REFERENCES

The publications and other materials listed below and/or set forth by author and date in the text above to illuminate the presently disclosed subject matter, and in particular cases, to provide additional details respecting the practice, are incorporated herein by reference. Materials used herein include but are not limited to the following listed references.
Adelman et al. (1983). DNA 2:183.
Altschul et al. (1990). J Mol Biol 215:403-410.
Andersson et al. (2000). Biopolymers 55:227-250.
Anguita et al. (2002). Immunity 16:849-859.
Berkow et al. (1997). The Merck Manual of Medical Information, Home ed. Merck Research Laboratories, Whitehouse Station, N.J.
Bodanszky (1993). Principles of Peptide Synthesis, 2nd rev. ed. Springer-Verlag, Berlin/New York.
Burgdorfer et al. (1982). Science 216: 1317-1319.
Chen et al. (1994). J. Clin. Microbiol. 32:589-595.
Corringer et al. (1993). J. Med. Chem. 36:166-172.
Daix et al. (2007). Insect Mol Biol. 16:155-166.
Das et al. (2001). J Infect Dis 184: 1056-1064.
Duch et al. (1998). Toxicol. Left. 100-101:255-263.
Ebadi (1998). CRC Desk Reference of Clinical Pharmacology. CRC Press, Boca Raton, Fla.
Fearon et al. (1975). J Exp Med. 142: 856-863.
Fearon. (1979). Proc Natl Acad Sci USA. 76: 5867-5871.
Ferreira and Silva (1998). Vet. Immunol. Immunopathol. 64:279-293.
Fields and Noble (1990). Int J Pept Protein Res 35:161-214.
Freireich et al. (1966). Cancer Chemothe.r Rep. 50:219-244.
Fujita et al. (1987). J Exp Med. 166: 1221-1228.
Garbay-Jaureguiberry et al. (1992). Int J Pept Protein Res 39:523-527.
Goodman et al. (1996). Goodman & Gilman's the Pharmacological Basis of Therapeutics, 9th ed. McGraw-Hill Health Professions Division, New York. Goundis et al. (1988). Nature. 335: 82-85.
Gribskov et al. (1986). Nuc Acids Res 14(1):327-334.
Guo et al. (1992). Proc Natl Acad Sci USA. 89:3040-3044.
Gupta-Bansal et al. (2000). Mol Immunol. 37: 191-201.
Holt et al. (1990). J Biol Chem. 265: 2852-2855.
Hourcade (2006). J Biol Chem 281(4):2128-2132.
Katzung (2001). Basic & Clinical Pharmacology, 8th ed. Lange Medical Books/McGraw-Hill Medical Pub. Division, New York.
Kopecky and Kuthejlova (1998). Parasite Immunol. 20:69-74.
Kyte et al. (1982). J Mol Biol 157:105.
Lawrie et al. (2005). Mol Immunol. 42: 31-38.
McOmie (1973). Protective Groups in Organic Chemistry, Plenum Press, London/New York.
McRae et al. (2005). J Immunol. 174: 6250-6256.
Merrifield (1969). Adv. Enzymol. Relat. Areas Mol. Biol. 32:221-296.
Morgan at al. (1999). Complement regulatory proteins. Academic Press, San Diego, Calif.
Motameni et al. (2004). Infect. Immun. 72:3638-3642.
Needleman et al. (1970). J Mol Biol 48:443.
Nicholson-Weller et al. (1982). J Immunol. 129: 184-189.
Ohnishi et al. (2001). Proc Natl Acad Sci USA 98: 670-675.
Pangburn et al. (1977). J Exp Med. 146: 257-270.
Pangburn et al. (1981). J Exp Med. 154: 856-867.
Pangburn et al. (1986). Biochem J. 235: 723-730.
Pavone et al. (1993). Int J Pept Protein Res 41:15-20.
Perdikoulis et al. (2001). Biochim Biophys Acta. 1548: 265-277.
PCT International Publication No. WO 93/25521.
Remington et al. (1975). Remington's Pharmaceutical Sciences, 15th ed. Mack Pub. Co., Easton, Pa.
Ribeiro at al. (1995). Biochem. J. 308:243-249.
Ribeiro at al. (2006) Insect Biochem Mol Biol 36:111-129.
Ross et al. (1982). J Immunol. 129: 2051-2060.
Sambrook et al. (2001). Molecular Cloning: A Laboratory Manual, Third Edition, Cold Spring Harbor Laboratory Press, Cold Spring Harbor, N.Y.
Schneider and Eberle (1993). Peptides, 1992: Proceedings of the Twenty-Second European Peptide Symposium, Sep. 13-19, 1992, Interlaken, Switzerland. Escom, Leiden.
Schoeler et al. (1999). Exp. Parasitol. 92:239-248.
Schröder and Lübke (1965). The Peptides. Academic Press, New York, United States of America.
Schwartz et al. (1979). Nuc Acids Res 6(2):745-755.
Smith et al. (1981). Adv Appl Math 2:482.
Speight et al. (1997). Avery's Drug Treatment: A Guide to the Properties, Choice, Therapeutic Use and Economic Value of Drugs in Disease Management, 4th ed. Adis International, Auckland/Philadelphia.
Spitzer et al. (2007). J Immunol. 179: 2600-2608.
Soares et al. (2005) Insect Mol Biol 14:443-452.
Sonenshine, D. E. (1993). Biology of Ticks. New York: Oxford University Press.
Stewart and Young (1969). Solid Phase Peptide Synthesis, Freeman, San Francisco.
Tatchell, R. J. (1967). J Parasitol 53: 1106-1107.
Thompson et al. (1994), Nucleic Acids Res 22(22):4673-4680.
Tucker, R. P. (2004). Int J Biochem Cell Biol. 36: 969-974.
Tung et al. (1992). Pept. Res. 5:115-118.
Tyson et al. (2007). Insect Mol Biol 16(4):469-479.
Tsao et al. (2006). Biochem Biophys Res Commun. 339: 779-784.
Urge et al. (1992). Carbohydr. Res. 235:83-93.
Urioste et al. (1994). J. Exp. Med. 180:1077-1085.
U.S. Pat. No. 4,244,946.
U.S. Pat. No. 4,554,101.
U.S. Pat. No. 5,234,933.
U.S. Pat. No. 5,326,902.
U.S. Pat. No. 5,578,629.
U.S. Pat. No. 5,811,392.
U.S. Pat. No. 5,811,512.
U.S. Pat. No. 5,811,515.
U.S. Pat. No. 5,817,757.
U.S. Pat. No. 5,817,879.
U.S. Pat. No. 6,015,561.
U.S. Pat. No. 6,015,881.
U.S. Pat. No. 6,031,071.
U.S. Pat. No. 6,180,082.
Valenzuela et al. (2000). J Biol Chem. 275: 18717-18723.
Walport (2001). N Engl J Med. 344: 1058-1066.
Weiler et al. (1976). Proc Natl Acad Sci USA. 73: 3268-3272.
Wethmur & Davidson (1968). J Mol Biol 31:349-370.
Whaley et al. (1976). J Exp Med. 144: 1147-1163.
Wikel and Bergman (1997). Parasitol. Today 13:383-389.
Zipfel et al. (1999). Immunopharmacology. 42: 53-60.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. An isolated and purified ILP family polypeptide, comprising:
(a) a polypeptide encoded by a nucleic acid sequence of any of odd numbered SEQ ID NOs: 1-30;
(b) a polypeptide encoded by a nucleic acid having at least about 90% or greater sequence identity to a DNA sequence of any of odd numbered SEQ ID NOs: 1-30;
(c) a polypeptide having an amino acid sequence of any of even numbered SEQ ID NOs: 1-30; or
(d) a polypeptide having an amino acid sequence having at least about 90% or greater sequence identity to an amino acid sequence of any of even numbered SEQ ID NOs: 1-30.

2. The polypeptide of claim 1, modified to be in detectably labeled form.

3. A composition comprising the polypeptide of claim 1 and a carrier.

4. The composition of claim 3, wherein the carrier is a pharmaceutically acceptable carrier.

5. An isolated nucleic acid molecule, comprising:
(a) a nucleic acid molecule encoding a polypeptide of any of even numbered SEQ ID NOs: 1-30;
(b) a nucleic acid molecule encoding a polypeptide having at least about 90% or greater sequence identity to a polypeptide of any of even numbered SEQ ID NOs: 1-30;
(c) a nucleic acid molecule having at least about 90% or greater sequence identity to a nucleic acid sequence of any of odd numbered SEQ ID NOs: 1-30; or
(d) a nucleic acid molecule having a nucleic acid sequence of any of odd numbered SEQ ID NOs: 1-30.

6. A recombinant vector comprising the nucleic acid molecule of claim 5 operatively linked to a promoter.

7. A recombinant host cell comprising the nucleic acid molecule of claim 5.

8. A method of modulating the activity of a protein having thrombospondin repeats, comprising contacting the protein having thrombospondin repeats with an ILP family protein comprising:
(a) a polypeptide encoded by a nucleic acid sequence of any of odd numbered SEQ ID NOs: 1-36;
(b) a polypeptide encoded by a nucleic acid having at least about 90% or greater sequence identity to a DNA sequence of any of odd numbered SEQ ID NOs: 1-36;
(c) a polypeptide having an amino acid sequence of any of even numbered SEQ ID NOs: 1-36; or
(d) a polypeptide having an amino acid sequence having at least about 90% or greater sequence identity to an amino acid sequence of any of even numbered SEQ ID NOs: 1-36,
wherein activity of the protein having thrombospondin repeats is modulated.

9. The method of claim 8, wherein the protein having thrombospondin repeats is properdin.

10. The method of claim 8, wherein the thrombospondin repeats are type 1 thrombospondin repeats.

11. The method of claim 8, wherein the protein having thrombospondin repeats is selected from a protein involved in cancer, homeostasis and pathogenesis.

12. The method of claim 8, wherein the protein having thrombospondin repeats is within a subject and the ILP family protein is administered to the subject.

13. The method of claim 12, wherein the ILP family protein is administered by systemic administration, parenteral administration, intravascular administration, intramuscular administration, intraarterial administration, oral delivery, buccal delivery, subcutaneous administration, inhalation, intratracheal installation, surgical implantation, transdermal delivery, local injection, hyper-velocity injection/bombardment, or combinations thereof.

14. A method of modulating the alternative complement pathway in a subject, comprising administering to the subject an effective amount of an ILP family protein comprising:
(a) a polypeptide encoded by a nucleic acid sequence of any of odd numbered SEQ ID NOs: 1-36;
(b) a polypeptide encoded by a nucleic acid having at least about 90% or greater sequence identity to a DNA sequence of any of odd numbered SEQ ID NOs: 1-36;
(c) a polypeptide having an amino acid sequence of any of even numbered SEQ ID NOs: 1-36; or
(d) a polypeptide having an amino acid sequence having at least about 90% or greater sequence identity to an amino acid sequence of any of even numbered SEQ ID NOs: 1-36,
wherein the alternative complement pathway is modulated.

15. The method of claim 14, wherein modulating the alternative complement pathway comprises reducing the activity of the alternative complement pathway.

16. The method of claim 15, wherein reducing the activity of the alternative complement pathway comprises the binding of the ILP family protein to properdin thereby accelerating the decay of the C3 convertase and reducing the activity of the alternative complement pathway.

17. The method of claim 16, wherein the ILP family protein binds to properdin by binding to the thrombospondin repeats on properdin.

18. The method of claim 14, wherein the ILP family protein is administered by systemic administration, parenteral administration, intravascular administration, intramuscular administration, intraarterial administration, oral delivery, buccal delivery, subcutaneous administration, inhalation, intratracheal installation, surgical implantation, transdermal delivery, local injection, hyper-velocity injection/bombardment, or combinations thereof.

19. The method of claim 14, wherein the subject is suffering from a condition associated with inappropriate alternative complement pathway activation.

20. The method of claim 19, wherein the condition associated with inappropriate complement pathway activation is selected from inflammatory diseases, arthritis, asthma, acute injuries, burns, heart disease, autoimmune diseases and SARS.

21. A method of treating a complication associated with inappropriate alternative complement pathway activation in a subject, comprising administering to the subject an effective amount of an ILP family protein comprising:
(a) a polypeptide encoded by a nucleic acid sequence of any of odd numbered SEQ ID NOs: 1-36;
(b) a polypeptide encoded by a nucleic acid having at least about 90% or greater sequence identity to a DNA sequence of any of odd numbered SEQ ID NOs: 1-36;
(c) a polypeptide having an amino acid sequence of any of even numbered SEQ ID NOs: 1-36; or
(d) a polypeptide having an amino acid sequence having at least about 90% or greater sequence identity to an amino acid sequence of any of even numbered SEQ ID NOs: 1-36,
wherein the complication is treated.

22. The method of claim 21, wherein treating a complication associated with inappropriate alternative complement pathway activation comprises reducing the activity of the alternative complement pathway.

23. The method of claim 22, wherein reducing the activity of the alternative complement pathway comprises binding of the ILP family protein to properdin thereby accelerating the decay of the C3 convertase and reducing the activity of the alternative complement pathway.

24. The method of claim 23, wherein the ILP family protein binds to properdin by binding to the thrombospondin repeats on properdin.

25. The method of claim 21, wherein the ILP family protein is administered by systemic administration, parenteral administration, intravascular administration, intramuscular administration, intraarterial administration, oral delivery, buccal delivery, subcutaneous administration, inhalation, intratracheal installation, surgical implantation, transdermal delivery, local injection, hyper-velocity injection/bombardment, or combinations thereof.

26. The method of claim 21, wherein the complication associated with inappropriate alternative complement pathway activation is selected from inflammatory diseases, arthritis, asthma, acute injuries, burns, heart disease, autoimmune diseases and SARS.

27. A method of screening a candidate substance for an ability to bind properdin, the method comprising:
(a) establishing a test sample comprising properdin;
(b) administering a candidate substance to the test sample; and
(c) determining the ability of the candidate substance to bind to properdin.

28. The method of claim 27, further comprising administering an ILP family protein to the test sample in step (b) and determining the ability of the candidate substance to bind to properdin based upon the competition between the candidate substance and the ILP family protein in step (c).

29. A method of screening for substances capable of modulating the activity of the alternative complement pathway, comprising:
(a) establishing a test sample comprising a protein having thrombospondin repeats;
(b) administering a candidate substance to the test sample;
(c) determining the ability of the candidate substance to bind to the protein having thrombospondin repeats; and
(d) identifying a candidate substance as capable of inhibiting the alternative complement pathway where the candidate substance is capable of binding to the protein having thrombospondin repeats.

30. The method of claim 29, wherein the protein having thrombospondin repeats is properdin.

31. The method of claim 29, further comprising administering an ILP family protein to the test sample in step (b) and determining the ability of the candidate substance to bind to the protein having thrombospondin repeats based upon the competition between the candidate substance and the ILP family protein in step (c).


SEQUENCE LISTING (40 sequences)

SEQ ID NO: 1 - DNA, 555 bp, Ixodes scapularis
atgaggactgcgttcacctgtgcgcttttggcgatttcgtttttaggaagcccgtgttcgtccagcgaagacggtctcgagcaagtttccaaagtggaaactactacacaaaatctctacgaacgtcattatagaaacaatcctggattgtgtgaggcacagtataggaattcaagccatgcggaagccgtttacaactgcacgctcagtcatttgcccccagtcctgaatgaaacctgggaaggaattaggcatcgaattaataaaagcatacctgagttcgtaagcttgatgtgcaactttactgttgcgatgcctaatgaattctacttagtttatatggggtcaaatggaaactcagactttgaagaggacaaagggagcacaggcactgatgaagacagtaacacgggatcttctgcggcagctaaacttacagaagagctaataatagaagcagaggtaaactgcacggcgcatataactggttggaccactgaagccccgaccacgctggaacctacgacagggcctcaatttgaggaaattccctga

SEQ ID NO: 2 - protein, 184 aa, Ixodes scapularis
MRTAFTCALLAISFLGSPCSSSEDGLEQVSKVETTTQNLYERHYRNNPGLCEAQYRNSSHAEAVYNCTLSHLPPVLNETWEGIRHRINKSIPEFVSLMCNFTVAMPNEFYLVYMGSNGNSDFEEDKGSTGTDEDSNTGSSAAAKLTEELIIEAEVNCTAHITGWTTEAPTTLEPTTGPQFEEIP

SEQ ID NO: 3 - DNA, 531 bp, Ixodes scapularis
atgaggactgtgctgacttgtgcacttttggcgattttatttctaggaagcccgtgttcgtccagcgaggacggtggagagcaagggtcacgagttgaaactactacacatagtctttacgaacgtcagtacagaaatcattctggattgtgcggggcacagtacaggaattccagctatgcggaacccgtttacaactgcacgcttaaagctttgcccccaatcgtaaataaaacctgggagggaattaggcataacattaggaagaccataccccagtttgtaagcttgatgtgcaacttcactgtggttatgcctgaaagcttctacttactttatatgggggaggcaacgtcaaactccgaagaggaggaagagagcacaggcactaccgaagagtctactgctgtgaaagttacaggacagctaataacagaagcagagaacgcctgcacggcgaatataactggttggacccctccaaccacgccggaaccgacgaagtcgcttgagcctgtggccgtcccctga

SEQ ID NO: 4 - protein, 176 aa, Ixodes scapularis
MRTVLTCALLAILFLGSPCSSSEDGGEQGSRVETTTHSLYERQYRNHSGLCGAQYRNSSYAEPVYNCTLKALPPIVNKTWEGIRHNIRKTIPQFVSLMCNFTVVMPESFYLLYMGEATSNSEEEEESTGTTEESTAVKVTGQLITEAENACTANITGWTPPTTPEPTKSLEPVAVP

SEQ ID NO: 5 - DNA, 525 bp, Ixodes scapularis
atgaggactgcgttcacctgtgcgcttttggcgatttcgtttctaggaagcccgtgttcgtccagcgaagacggtctcgagcaagataccaaagtggaaactactacacaaaatctctacgaacgtcattatagaaacaatcctggattgtgcggggcacagtataggaattcaagccatgcggaagccgtttgcaactgcacgctcaatcatttgcccccagtcgtgaatacaacctgggaaagaattaggcatcgaattaacaaaaccatacctgagttcgtaaggttgatgtgcaactttactgttgcgatgcctgatgacttctacttagtttatatggggtcaaatggaaactcaaactctgaagagggcaatgagagcacagacactgataacgaagttacagaacagctaatattaaaagcagaggaaaactgcacggcgcatataactggttggaccactgaagccccgaccacgctggaaccgacgaagtcgcttgagcctgtggccgtcccctga

SEQ ID NO: 6 - protein, 174 aa, Ixodes scapularis
MRTAFTCALLAISFLGSPCSSSEDGLEQDTKVETTTQNLYERHYRNNPGLCGAQYRNSSHAEAVCNCTLNHLPPVVNTTWERIRHRINKTIPEFVRLMCNFTVAMPDDFYLVYMGSNGNSNSEEGNESTDTDNEVTEQLILKAEENCTAHITGWTTEAPTTLEPTKSLEPVAVP

SEQ ID NO: 7 - DNA, 525 bp, Ixodes scapularis
atgaggactgcgttcacctgtgcgcttttggcgatttcgtttctaggaagcccgcgttcgtccagcgaagacggtctcgagcaagataccaaagtggaaactactacacaaaatctctacgaacgtcattatagaaacaatcctggattgtgcggggcacagtataggaattcaagccatgcggaagccgtttacaactgcacgctcaatcatttgcccccagtcgtgaatacaacctgggaaagaattaggcatcgaattaacaaaaccatacctgagttcgtaaggttgatgtgcaactttactgttgcgatgcctgatgacttctacttagtttatatggggtcaaatggaaactcaaactctgaagagggcaatgagagcacagacactgataacgaagttacagaacagctaatattaaaagcagaggaaaactgcacggcgcatataactggttggaccactgaagccccgaccacgctggaacctacgacggagcctcaatttaaggaaatttcctga

SEQ ID NO: 8 - protein, 174 aa, Ixodes scapularis
MRTAFTCALLAISFLGSPRSSSEDGLEQDTKVETTTQNLYERHYRNNPGLCGAQYRNSSHAEAVYNCTLNHLPPVVNTTWERIRHRINKTIPEFVRLMCNFTVAMPDDFYLVYMGSNGNSNSEEGNESTDTDNEVTEQLILKAEENCTAHITGWTTEAPTTLEPTTEPQFKEIS

SEQ ID NO: 9 - DNA, 532 bp, Ixodes scapularis
atgaggactgcgctcacctgtgcgcttttggcgatttcgtttctaggaagcccgtgttcgtcaagcgaaggcggtctcgagaaagattccagagtggaaactactacacaaaatctctacgaacgttattatagaaaacatcctggattgtgcggggcacagtataggaattcaagccatgcggaagccgtttacaactgcacgctcaatcatttgcccccagtcctgaatgaaacctgggaaggaattaggcatcgaattaataaaagcatacctgagttcgtaagcttgatgtgcaattttactgttgcgatgcctgacgacttctacttagcttatatggggtcaaatggaaactcaaactctgaagaggacgaagagagcacagacactgctaaccaagttacagaagagctattaacaaaagcagaggaaaactgcacggcgcatataactggttggaccactgaagccccgagtcccgaccacgctggaacctacgacggagactcaattcgaggcaatttcctga

SEQ ID NO: 10 - protein, 177 aa, Ixodes scapularis
MRTALTCALLAISFLGSPCSSSEGGLEKDSRVETTTQNLYERYYRKHPGLCGAQYRNSSHAEAVYNCTLNHLPPVLNETWEGIRHRINKSIPEFVSLMCNFTVAMPDDFYLAYMGSNGNSNSEEDEESTDTANQVTEELLTKAEENCTAHITGWTTEAPSPDHAGTYDGDSIRGNFL

SEQ ID NO: 11 - DNA, 525 bp, Ixodes scapularis
atgaggactgcgctcacctgtgcgcttttggcgatttcgtttctaggaagcccgtgttcgtccagcgaagacggtctagagcaagattccaaagtggaaactactacacaaaatctctacgaacgtcattatagaaacaatcctggattgtgcggggcacagtataggaattcaagccatgcggaagccgtttacaactgcacgctccatcttttgcccccagtcgtgaatacaacctgggaaggaattaagcatcgaattaacaaaaccatacctgagtttgtaaatttgatttgcaacttaactgttgcgatgcctgatgagtcctacttagtttatatggggtcagatggaaacacaaactctgaagaggacaaggagagcacagacactgataaagaagttacagaaaagctaataataaaagcaggggaaaactgcacggcgcatataactggttggaccactgaagccccgaccacgctggaacctacgacggagactcaattcgaggcaatttcctga

SEQ ID NO: 12 - protein, 174 aa, Ixodes scapularis
MRTALTCALLAISFLGSPCSSSEDGLEQDSKVETTTQNLYERHYRNNPGLCGAQYRNSSHAEAVYNCTLHLLPPVVNTTWEGIKHRINKTIPEFVNLICNLTVAMPDESYLVYMGSDGNTNSEEDKESTDTDKEVTEKLIIKAGENCTAHITGWTTEAPTTLEPTTETQFEAIS

SEQ ID NO: 13 - DNA, 525 bp, Ixodes scapularis
atgaggactgcgctcacctgtgcgcttttggcgatttcgtttctaggaagcccgtgttcgtcaagcgaaggcggtctcgagaaagattccagagtggaaactactacacaaaatctctacgaacgttattatagaaaacatcctggattgtgcggggcacagtataggaattcaagccatgcggaagccgtttacaactgcacgctccatcttttgcccccagtcgtgaatacaacctgggaaggaattaggcatcgaattaacaaaaccatacctgagtttgtaaatttgatttgcaacttaactgttgcgatgcctgatgagttctacttagtttatatggggtcagatggaaacacaaactctgaagaggacaaggagagcacagacactgataaaggagttacagaaaagctattaacagaagcagaggaaaactgcacggcgcatataactggttggaccactgaagccccgaccacgctggaacctacgacggagactcaattcgaggcaatttcctga

SEQ ID NO: 14 - protein, 174 aa, Ixodes scapularis
MRTALTCALLAISFLGSPCSSSEGGLEKDSRVETTTQNLYERYYRKHPGLCGAQYRNSSHAEAVYNCTLHLLPPVVNTTWEGIRHRINKTIPEFVNLICNLTVAMPDEFYLVYMGSDGNTNSEEDKESTDTDKGVTEKLLTEAEENCTAHITGWTTEAPTTLEPTTETQFEAIS

SEQ ID NO: 15 - DNA, 522 bp, Ixodes scapularis
atgaggactgcgttcacctgtgcgcttttggcgatttcgtttctaggaagcccgtgttcgtccagcgaagacggtctcgagcaagattccaaagtggaaactacaacacaaaatctctacgaacgtcaatatagaaaccattctggattgtgcggggcacagtataggaattcaagtcatgcggaagccgtttacaactgcacgctcaatcttttgcccccagtcgtgaatgcaacctgggaaggaattaggcatcgaattaataaaaccatacctcagtttgtaaaattgatttgcaactttactgttgcgatgcctgatgacttccgcttagtttatatggggtcaaatggaaactcaaactctgaagaggacaaagagagcacagacactggtaagcaagttacagcagagctaataatgaaagcagaggaaaactgcacggcgcatataactggttggaccactgaagccccgaccacgctggaacctacagagtctgaatataaggaaatttcgtga

SEQ ID NO: 16 - protein, 173 aa, Ixodes scapularis
MRTAFTCALLAISFLGSPCSSSEDGLEQDSKVETTTQNLYERQYRNHSGLCGAQYRNSSHAEAVYNCTLNLLPPVVNATWEGIRHRINKTIPQFVKLICNFTVAMPDDFRLVYMGSNGNSNSEEDKESTDTGKQVTAELIMKAEENCTAHITGWTTEAPTTLEPTESEYKEIS

SEQ ID NO: 17 - DNA, 525 bp, Ixodes scapularis
atgaggactgcgctcacctgtgcgcttttggcgatttcgtttctaggaagcccgtgttcgtcaagcgaaggcggtctcgagaaagattccagagtggaaactacaacacaaaatctctacgaacgtcattatagaaatcattctggattgtgcggggcacagtataggaattcaagccatgcggaagccgtttacaactgcacgctcaatcttttgcccccagtcgtgaatgcaacctgggaaggaattaggcatcgaattaataaaaccatacctcagtttgtaaaattgatttgcaactttactgttgcgatgcctgatgacttccacttagtttatatggggtcagatggaaactcaaactctgaagaggacaaggagagcacagacactgatgaagaagttacacaagagctaataataaaagcagaggaaaactgcacggcgcatataactggttggaccactgaagccccgaccacgccggaacctacgacagagcctcaatttgacgaaattccctga

SEQ ID NO: 18 - protein, 174 aa, Ixodes scapularis
MRTALTCALLAISFLGSPCSSSEGGLEKDSRVETTTQNLYERHYRNHSGLCGAQYRNSSHAEAVYNCTLNLLPPVVNATWEGIRHRINKTIPQFVKLICNFTVAMPDDFHLVYMGSDGNSNSEEDKESTDTDEEVTQELIIKAEENCTAHITGWTTEAPTTPEPTTEPQFDEIP

SEQ ID NO: 19 - DNA, 525 bp, Ixodes scapularis
atgaggactgcgttcacctgtgcgcttttggcgatttcgtttctaggaagcccgtgttcgtccagcgaagacagtctcgagcaagataccaaagtggaaactactacacaaaatctctacgaacgtcattatagaaacagtcctggattgtgcggggcacagtataggaattcaagccatgcggaagccgtttacaactgcacgctcaatcatttgcccccagtcctgaatgaaacctgggaaggaattaggcatcgaattaataaaagcatacctgagttcgtaagcttgatgtgcaactttactgttgcgatgcctcatgaattctacttagcttatatggggtcaaatggaaactcaaactctgaagaggacgaagagagcacagacactgctaaccaagttacagaagagctattaacaaaagcagaggaaaactgcacggcgcatataactggttggaccactgaagccccgaccacgctggaacctacgacggagcctcaatttgaggcaatttcctga

SEQ ID NO: 20 - protein, 174 aa, Ixodes scapularis
MRTAFTCALLAISFLGSPCSSSEDSLEQDTKVETTTQNLYERHYRNSPGLCGAQYRNSSHAEAVYNCTLNHLPPVLNETWEGIRHRINKSIPEFVSLMCNFTVAMPHEFYLAYMGSNGNSNSEEDEESTDTANQVTEELLTKAEENCTAHITGWTTEAPTTLEPTTEPQFEAIS

SEQ ID NO: 21 - DNA, 525 bp, Ixodes scapularis
atgaggactgcgctcacctgtgcgcttttggcgatttcgtttctaggaggcccgtgtttgtccagcgaagacggtctcgagcaaggttccattgtggaaactactacacaaaatctctacgaacgtcattatagaaatcatcctggattgtgcggggcacagtataggaattcaagccatgcggaatccatttacaactgcacacttcgtcttttgcccccaatcgtgaatcaaacctgggaaagaattaggcataaaattaataggaccatacctgattttgtaaaattgatttgcaactttactgttgcgatgcctgacgacttctacttcgtttatttggggtcaaatggaaactcagactttgaaggggacgaagagggcacagacactgataaagaagttacaggagagctaataatgaaagcagaggaaaactgcacggcgcatataactggttggaccactgaagccccgaccacgctggaacctacgacggagactcaattcgaggcaatttcctga

SEQ ID NO: 22 - protein, 174 aa, Ixodes scapularis
MRTALTCALLAISFLGGPCLSSEDGLEQGSIVETTTQNLYERHYRNHPGLCGAQYRNSSHAESIYNCTLRLLPPIVNQTWERIRHKINRTIPDFVKLICNFTVAMPDDFYFVYLGSNGNSDFEGDEEGTDTDKEVTGELIMKAEENCTAHITGWTTEAPTTLEPTTETQFEAIS

SEQ ID NO: 23 - DNA, 552 bp, Ixodes scapularis
atgaggactgcgctcacctgtgcgcttttggcgatttcgtttctaggaagcccgtgttcgtccagcgaagacggtctagagcaagattccaaagtggaaactactacacaaaatctctacgaacgtcattatagaaataattctggattgtgcggggcacagtataggaattcaagccatgcggaagccgtttacaactgcacgctcagtcttttgcccccaaaggtgaatacaacctgggaagaaattaggcatcgaattaataaaagcatacctgagtttgtaaggtcgatctgcaactttactgttgcgatgcctatggacttctactcagtttatatggggtcaaatggaaactcatactctgaagaggacgaagagagcacagacactgacaaagacagtaaaacggggtcttctgctgcagttgaagttacagaaaagctaataatggaagcagaggaaaactgcacggcgcatataactggttggaccactgaagccccgaccacgctggaacctacggagactcaattcgaggcaatttcctga

SEQ ID NO: 24 - protein, 183 aa, Ixodes scapularis
MRTALTCALLAISFLGSPCSSSEDGLEQDSKVETTTQNLYERHYRNNSGLCGAQYRNSSHAEAVYNCTLSLLPPKVNTTWEEIRHRINKSIPEFVRSICNFTVAMPMDFYSVYMGSNGNSYSEEDEESTDTDKDSKTGSSAAVEVTEKLIMEAEENCTAHITGWTTEAPTTLEPTETQFEAIS

SEQ ID NO: 25 - DNA, 525 bp, Ixodes scapularis
atgaggactgcgttcacctgtgcgcttttggcgatttcgtttctaggaagcccgtgttcgtccagcgaagacggtctcgagcaagataccaaagtggaaactactacacaaaatctctacgaacgtcattataggaacaatcctggattgtgcggggcacagtataggaattcaagccatgcggaagccgtttacaactgcacgctcagtcttttgcccccaagggtgaatacaacctgggaaggaattaggcatcgaattaacaaaaccatacctgagtttgtaaaattgatttgcaactttactgttgcggtgcctgatgagttccacttagtttatatggggtcaaatggaaactcaaactctgaaaaggacgaagagagcacagacactgataaccaagttacagaagagctattaacaaaagcagaggaaaactgcacggcgcatataactggatggaccactgaagccccgaccacgctggaacctacgacggagcctcaatatgaggcaatttcttga

SEQ ID NO: 26 - protein, 174 aa, Ixodes scapularis
MRTAFTCALLAISFLGSPCSSSEDGLEQDTKVETTTQNLYERHYRNNPGLCGAQYRNSSHAEAVYNCTLSLLPPRVNTTWEGIRHRINKTIPEFVKLICNFTVAVPDEFHLVYMGSNGNSNSEKDEESTDTDNQVTEELLTKAEENCTAHITGWTTEAPTTLEPTTEPQYEAIS

SEQ ID NO: 27 - DNA, 555 bp, Ixodes scapularis
atgaggactgcgttcacctgtgcgcttttggcgatttcgtttcttggaagcccgtgttcgtccagcgaagacggtctcgagcaaggttccattgtggaaactactacacaaaatctctacgaacgttattatagaaatcattctggattgtgcggggcacagtataggaattcaagccatgcggaatccatttacaactgcacgcttcgtcttttgcccccaatcgtgaatcaaacctgggaaagaattaggcatagaattaataggaccatacctgcatttgtaaaattgatttgcaacttgactgttgcgatgcctgacgacttctacttagtttatttggggtcaaatggaaactcagactttgaaggggacgaagagggcacagacactggtaaaggcggtaaaacggggtcttctgctgcagttcaagttacagaacagctaataacacaagcagaggaaaactgcaccgcgcaaataactggttggaccactgaagccccgaccacgctggaacctacgacggagcctgaattagaggcaattccctga

SEQ ID NO: 28 - protein, 184 aa, Ixodes scapularis
MRTAFTCALLAISFLGSPCSSSEDGLEQGSIVETTTQNLYERYYRNHSGLCGAQYRNSSHAESIYNCTLRLLPPIVNQTWERIRHRINRTIPAFVKLICNLTVAMPDDFYLVYLGSNGNSDFEGDEEGTDTGKGGKTGSSAAVQVTEQLITQAEENCTAQITGWTTEAPTTLEPTTEPELEAIP

SEQ ID NO: 29 - DNA, 534 bp, Ixodes scapularis
atgaggactgcgctcacctgtgcgcttttggcgatttcgtttctaggaagcccgtgttcgtcaagcgaaggcggtctcgagaaagattccagagtggaaactactacacaaaatctctacgaacgttattatagaaaacatcctggattgtgcggggcacagtataggaattcaagccatgcggaagccgtttacaactgcacgctcagtcttttgcccctaagcgtgaatacaacctgggaaggaattaggcatcgaattaacaaaaccatacctgagtttgtaaatttgatttgcaactttactgttgcgatgcctgatcagttctacttagtttatatggggtcaaatggaaactcatactctgaagaggacgaagacggtaaaaccgggtcttctgctgcagttcaagttacagagcagctaataatacaagcagaggaaaactgcacggcgcatataactggttggaccactgaagccccgaccacgctggaacctacgacggagactcaattcgaggcaatttcctga

SEQ ID NO: 30 - protein, 177 aa, Ixodes scapularis
MRTALTCALLAISFLGSPCSSSEGGLEKDSRVETTTQNLYERYYRKHPGLCGAQYRNSSHAEAVYNCTLSLLPLSVNTTWEGIRHRINKTIPEFVNLICNFTVAMPDQFYLVYMGSNGNSYSEEDEDGKTGSSAAVQVTEQLIIQAEENCTAHITGWTTEAPTTLEPTTETQFEAIS

SEQ ID NO: 31 - DNA, 555 bp, Ixodes scapularis
atgaggactgcgtttacctgtgctcttttggcgatttcgtttctaggaagcccgtgttcgtccagcgaagacggtctcgagcaagataccatagtggaaactactacacaaaatctctacgaacgtcattatagaaatcattctggattgtgcggggcacagtataggaattcaagccatgcggaagccgtttacaactgcacgctcaatcatttgcccccagtcgtgaatgcaacctgggaaggaattaggcatcgaattaataaaaccatacctcagtttgtaaaattgatttgcaactttactgttgcgatgcctcaagaattctacttagtttatatggggtcagatggaaactcagactttgaagaggacaaagagagcacaggcactgatgaagacagtaacacgggatcttctgctgcagctaaagttacagaagcgctaataatagaagcagaggaaaactgcacggcgcatataactggttggaccactgaaaccccgaccacgctggaacctacgacagagtctcaatttgaggcaattccctga

SEQ ID NO: 32 - protein, 184 aa, Ixodes scapularis
MRTAFTCALLAISFLGSPCSSSEDGLEQDTIVETTTQNLYERHYRNHSGLCGAQYRNSSHAEAVYNCTLNHLPPVVNATWEGIRHRINKTIPQFVKLICNFTVAMPQEFYLVYMGSDGNSDFEEDKESTGTDEDSNTGSSAAAKVTEALIIEAEENCTAHITGWTTETPTTLEPTTESQFEAIP

SEQ ID NO: 33 - DNA, 246 bp, Ixodes scapularis
atgtgcaacttcactgttgctatgcctgataacttctacttactttatatgggggacgcaacgtcaaactacgactccgaagaggaggaacagagcacaggcactactgaagagtctcctgctgtgaaagttacagaacagcaaataacagacgcagagaacgcctgcacggcgaatataactggttggacccctccaaccacgccggaaccgacgaagtctcttgagcctgtggccgtcccctga

SEQ ID NO: 34 - protein, 81 aa, Ixodes scapularis
MCNFTVAMPDNFYLLYMGDATSNYDSEEEEQSTGTTEESPAVKVTEQQITDAENACTANITGWTPPTTPEPTKSLEPVAVP

SEQ ID NO: 35 - DNA, 552 bp, Ixodes scapularis
atgaggactgcgctcacctgtgcgcttttggcgatttcgtttctaggaagcccgtgttcgtccagcgaagacggtctagagcaagattccaaagtggaaactactacacaaaatctctacgaacgtcattatagaaataattctggattgtgcggggcacagtataggaattcaagccatgcggaagccgtttacaactgcacgctcagtcttttgcccccaaaggtgaatgaaacctgggaaggaattaggcatcgaattaataaaagcatacctgagttcgtaaggttgatgtgcaactttagtgttgtgatgcctgaagacttctacttagtttatatggggtcaaatggaaactcatactctgaagaggacgaagagagcacagacactgacaaagacagtaaaacggggtcttctgctgcagttgaagttacagaaaagctaatattaaaagcagaggaaaactgcacggcgcatataactggttggaccactgaagccccgaccacgctggaacctacggagactcaattcgaggcaatttcctga

SEQ ID NO: 36 - protein, 183 aa, Ixodes scapularis
MRTALTCALLAISFLGSPCSSSEDGLEQDSKVETTTQNLYERHYRNNSGLCGAQYRNSSHAEAVYNCTLSLLPPKVNETWEGIRHRINKSIPEFVRLMCNFSVVMPEDFYLVYMGSNGNSYSEEDEESTDTDKDSKTGSSAAVEVTEKLILKAEENCTAHITGWTTEAPTTLEPTETQFEAIS

SEQ ID NO: 37 - DNA, 20 bp, Ixodes scapularis
ccagccatgaggactgcgct

SEQ ID NO: 38 - DNA, 24 bp, Ixodes scapularis
tcaggaaattgcctcgaattgagt

SEQ ID NO: 39 - DNA, 19 bp, Ixodes scapularis
cactgaggttcagagcaag

SEQ ID NO: 40 - DNA, 21 bp, Ixodes scapularis
gtatcagaactgtgcttgcac